(19) United States
(12) Patent Application Publication
(10) Pub. No.: US 2017/0191069 A1
(43) Pub. Date: Jul. 6, 2017

(54) ABORTIVE PROMOTER CASSETTES AND METHODS FOR FUSION TO TARGETS AND QUANTITATIVE CpG ISLAND METHYLATION DETECTION USING THE SAME

(71) Applicant: Ribomed Biotechnologies, Inc., San Diego, CA (US)

(72) Inventors: David McCarthy, San Diego, CA (US); Michelle M. Hanna, San Diego, CA (US)

(21) Appl. No.: 15/397,699

(22) Filed: Jan. 3, 2017

Related U.S. Application Data
(60) Provisional application No. 62/274,369, filed on Jan. 3, 2016.

(51) Int. Cl. C12N 15/64; C12Q 1/68

(52) U.S. Cl. CPC C12N 15/64; C12Q 1/6872; C12Q 1/686; C12Q 1/6827

(57) ABSTRACT

The present invention provides methods to assemble and fuse a full length Abortive Promoter Cassette (APC) to a target nucleic acid during PCR amplification of the target. The linked APC is used to quantify amplicon abundance by the production of RNA Abscripts from the synthetic APC. Stepwise PCR-dependent promoter assembly allows for target-fusion of APCs that are too long to be synthesized as monolithic promoter-primer oligonucleotide reagents.

### RELATED APPLICATIONS

This application claims the benefit of priority under 35 USC §119 of U.S. Provisional Application Ser. No. 62/274,369 filed Jan. 3, 2016, the entire disclosure of which is incorporated herein by reference.

### FIELD OF THE INVENTION

The invention generally pertains to the field of epigenomics and detection of diseases and conditions having an epigenomic basis. More specifically, the present invention relates to detection of DNA methylation, DNA methylation profiles and SNP detection, particularly in clinical samples.

### BACKGROUND

DNA methylation detection methods for genome level analysis and characterization of specific loci have had extensive use in biomarker discovery and cancer research. See e.g., Jones, Nat Rev Genet. 13:484-92 (2012); Noehammer et al., Epigenomics. 6:603-22 (2014); Kondo & Issa, Expert Rev Mol Med. 12:e23 (2010). There has been a slow translation of these methods into clinical tests. A major barrier is the need for high sensitivity when processing clinical samples. Most solid tumors are formalin fixed and paraffin embedded (FFPE) to allow WHO grading by a pathologist. The fixation process damages the DNA in the sample by causing DNA-DNA and DNA-protein crosslinks. DNA strand lengths are shortened due to the accumulation of strand breaks during fixation and upon long term storage. See van Beers, et al., Br J Cancer. 94:33-37 (2006). Sequence artifacts also are induced that cause conversion of cytosine to thymine. Do & Dobrovic, Oncotarget. 3:546-58 (2012); Do & Dobrovic, Clin Chem. 61:64-71 (2015); Do et al., Clin Chem. 59:1376-83 (2013). In spite of these problems, FFPE samples can be routinely analyzed in a research setting where there is access to relatively large samples. A significant sample rejection rate due to fixation damage can be tolerated as long as an adequate number of acceptable samples can be collected. Hegi et al., N Engl J Med. 352:997-1003 (2005). Clinical laboratories, however, must deal with limited sample sizes. A high rejection rate is unacceptable in clinical testing.

The most commonly used DNA methylation detection methods are based on sequence conversion by treatment with sodium-bisulfite. Clark et al., Nucleic Acids Res. 22:2990-97 (1994). Bisulfite causes the conversion of cytosine, but not 5-methyl-cytosine, to uracil. The resulting sequence difference can be detected by nucleotide sequencing or the use of PCR primers and probes that overlap with and discriminate between CpG and UpG. Diverse methods that rely on bisulfate treatment share common drawbacks when applied to clinical testing. DNA conversion induces damage, on top of the damage caused by fixation, causing reduced sensitivity and imposing a requirement for higher sample input. Sequence conversion is difficult to perform completely and reproducibly with FFPE samples. Genereux et al., Nucleic Acids Res. 36:e150 (2008) Tournier et al., BMC Cancer 12:12 (2012). Sample loss is possible during sample clean-up to remove residual bisulfite. Munson et al., Nucleic Acids Res. 35:2893-2903 (2007). The reduction in the amplifiable fraction after bisulfite treatment is large enough to require quality control measures for the treated DNA. Ehrich et al., Nucleic Acids: 35:e29 Res. (2007).

The bisulfite dependent methods MS-PCR and real time MS-PCR use probes that overlap with a small number of CpGs. An inference about the methylation level of a relatively large target is based on the success or failure of PCR amplification with each probe set. The accuracy of this approach is adversely affected by heterogeneity in a target where individual molecules have different methylation patterns. Probes that focus on one extreme or the other of the possible methylation patterns are unlikely to generate signals from divergent partially methylated target molecules. Methods that measure bisulfite induced changes in Tₘ, like DNA melting curve analysis, can detect methylation heterogeneity because all the CpG sites contribute to the Tₘ determination, however methylated DNA quantification is complicated from melt profiles of heterogeneous samples. Smith et al., BMC Cancer 9:123 (2009); Wojdacz & Dobrovic, Nucleic Acids Res. 35:e41 (2007).

Bisulfite-free methods avoid the complications associated with DNA sequence conversion. Methylation Sensitive Restriction Endonuclease (MRSE) methods exploit restriction sites containing CpG. MRSE methods rely on enzymes that cleave unmethylated targets but leave fully methylated targets unaffected. The uncleaved methylated DNAs are then amplified. This method is susceptible to incomplete digestion, which is especially acute with FFPE and damaged DNA samples. MRSE methods are also adversely affected by heterogeneity in the methylation pattern. Hashimoto et al., Epigenetics. 2:86-91 (2007); Melnikov et al., Nucleic Acids Res. 33: e93 (2005). For example, the presence of a persistently unmethylated targeted restriction site can cause an otherwise methylated DNA to appear unmethylated.

Applicants previously described a bisulfite and MSRE-free assay, MethylMeter (FIG. 1), which uses a Me-CpG-binding domain protein to separate methylated DNA from unmethylated DNA and a novel abortive transcription (Abscription) based signal generation process to measure methylation levels of specific targets. See U.S. Pat. No. 8,263,339, the contents of which is incorporated herein by reference in its entirety. Most methylated DNA affinity methods use the DNA binding domain of the MBD2 protein in different formats. MBD2 has the highest affinity for methylated DNA among related binding proteins MeCP2, MBD1 and MBD4 and is unaffected by the sequence context of a methylated CpG site. Fraga et al., Nucleic Acids Res. 31:1765-74 (2003); Hendrich & Bird, Mol Cell Biol. 18:6538-47 (1998); Klose et al., Mol Cell. 19:667-78 (2005). The recombinant MBD2 protein has been made in a hexa-His tagged form, which exists as a monomer, or as a GST-fusion protein, which exists as a dimeric MBD due to the dimerization of the GST domain. Fabrini et al., Biochemistry. 48:10473-82 (2009); Gebhard et al., Nucleic Acids Res. 34:e82 (2006). Dimerization is expected to increase affinity to methylated DNA based on results with concatemeric forms. Heimer et al., Protein Eng Des Sel. 28:543-51 (2015); Jorgensen et al., Nucleic Acids Res. 34:e96 (2006). An alternative approach in the MIRA method is to include MBD3L, which binds to MBD2 to make a high affinity complex. Rauch & Pfeifer, Methods Mol Biol. 507:65-75 (2009).

The Me-CpG binding protein, MethylMagnet (see U.S. Pat. No. 8,242,243), takes advantage of the high specificity of the MBD2 methyl-CpG binding domain and its bias for clustered CpGs, which favors the analysis of CpG islands. Fraga et al., Nucleic Acids Res. 31:1765-74 (2003); Serre et al., Nucleic Acids Res. 38:391-9 (2010). Because all methylated CpGs in a fragment can contribute to binding, the assay is less affected by heterogeneity of the methylation pattern. After separation, methylated and unmethylated targets are amplified and tagged for Abscription by PCR of each fraction. The PCR step plays no role in the discrimination between methylated and unmethylated targets so there are fewer constraints on primer development than there are for bisulfite based methods.

The high sensitivity of MethylMeter is achieved by using abortive transcription (Abscription) for detection in a process called CAP (Coupled Abscription PCR), which we have shown previously to be two to three orders of magnitude more sensitive than Taqman qPCR-based detection. McCarthy et al., “A quantitative, sensitive, and bisulfite-free method for analysis of DNA methylation.” (in DNA Methylation. Tatarinova & Kerton, eds., InTech, Rijeka. p. 93-116 (2012). Abscription involves the reiterative synthesis of short “abortive” RNA transcripts by an RNA polymerase without moving from the promoter. Johnston & McClure, “Abortive initiation of in vitro RNA synthesis on bacteriophage 1 DNA.” in RNA Polymerase. (Losick & Chamberlin, eds. Cold Spring Harbor Laboratory: Cold Spring Harbor, N.Y. p. 413-28 (1976)). Although this process occurs naturally at low turnover with many RNA polymerases, by manipulating the DNA promoter sequences, several Abortive Promoter Cassettes (APCs) have been developed, each designed to generate hundreds to thousands of copies of a single, specific, short abortive RNA transcript (Abscript) per minute. The APCs are attached to the amplicons generated from the DNA targets in the methylated and unmethylated fractions, and the amount of each Abscript produced is a measure of the amount of the original DNA target in each fraction. The signal amplification provided by Abscription reduces the number of PCR cycles required to detect even picograms of DNA to 29 to 31 cycles, reducing the non-specific amplification associated with higher cycle numbers.

### SUMMARY OF THE INVENTION

The present invention provides method for detecting CpG island methylation comprising the steps of separating methylated DNA comprising at least one CpG island from unmethylated DNA in a sample; and

performing Coupled Abscription PCR to detect the presence of the at least one CpG island nucleotide sequence in the methylated DNA, thereby detecting CpG island methylation. In certain aspects of the invention, the Coupled Abscription PCR uses three primers, which can be, for example, a forward target-specific primer that has a truncated Abortive Promoter Cassette (APC) sequence at its 5′ end; a reverse target-specific primer for creating an amplicon that contains a duplex inactive APC; and an APC primer that overlaps with the 5′ end of the truncated APC sequence. In other aspects of the invention the Coupled Abscription PCR uses four primers, which can be a forward target-specific primer that has a truncated APC sequence at its 5′ end; a reverse target-specific primer comprising a universal primer sequence at the 5′ end of a target-specific priming sequence; an APC completion primer that overlaps with the 5′ end of the truncated APC sequence; and a universal reverse primer.

In certain embodiments, the presence of the at least one CpG island nucleotide sequence is detected by fluorescence. For example, the fluorescence can be produced by opening of a molecular beacon, e.g., by an Abscript produced during Coupled Abscription PCR. In other embodiments, the presence of the at least one CpG island nucleotide sequence can be detected by mass spectrometry.

The CpG island may be an island that has been associated with cancer, including but not limited to a sequence selected from the group consisting of: SEQ ID NOs:39-52.

In other embodiments of the invention or more CpG island nucleotide sequences are detected by the methods of the invention. In certain aspects, at least two CpG island nucleotide sequences selected from the group consisting of: SEQ ID NOs: SEQ ID NOs:39-52 are detected. In other embodiments, at least two CpG island nucleotide sequences selected from the group consisting of: SEQ ID NOs:41-43 are detected.

Additional tests can be performed on the methylated or unmethylated fractions, such as detection of a Single Nucleotide Polymorphism (SNP) in the sample.

Also provided by the invention are methods for assembling and fusing a full length Abortive Promoter Cassette (APC) to a target nucleic acid during PCR amplification of the target comprising the steps of: a) providing a forward target-specific primer that has a truncated APC sequence at its 5′ end; b) providing a reverse target-specific primer for creating an amplicon that contains a duplex inactive APC; c) providing an APC primer that overlaps with the 5′ end of the truncated APC sequence; and d) amplifying the target with the three primers, thereby assembling and fusing a full-length APC to a target nucleic acid during PCR amplification of the target. In certain aspects, the primer of step a) is present at a lower concentration than primers of steps b) and c) during the amplifying step. The target can include, for example, a CpG island, which may be a sequence selected from the group consisting of: SEQ ID NOs:39-52.

Also provided by the invention are methods for assembling and fusing a full length Abortive Promoter Cassette (APC) to a target nucleic acid during PCR amplification of the target comprising the steps of: a) providing a forward target-specific primer that has a truncated APC sequence at its 5′ end; b) providing a reverse target-specific primer comprising a universal primer sequence at 5′ end of a target-specific priming sequence; c) providing an APC completion primer that overlaps with the 5′ end of the truncated APC sequence; d) providing a universal reverse primer; and e) amplifying the target with the four primers, thereby assembling and fusing a full-length APC to a target nucleic acid during PCR amplification of the target. In certain aspects, the primer of step a) is present at a lower concentration than primers of steps b) and c) during the amplifying step. The target can include, for example, a CpG island, which may be a sequence selected from the group consisting of: SEQ ID NOs:39-52.

### BRIEF DESCRIPTION OF THE DRAWINGS

FIGS. 1A and 1B are schematic diagrams illustrating the MethylMeter process. In FIG. 1A, fragmented DNA is purified into methylated and unmethylated fractions with the use of magnetic beads bearing a GST-MBD2 protein. The GST-MBD2 protein binds to DNA fragments with high densities of methylated-CpGs (1a). The presence of a targeted CpG island in either the methylated DNA fraction or the unmethylated fraction (depleted supernatant fraction) is measured by Coupled Abscription-PCR (CAP). The fragmented DNA sample can also be used for other assays such as single nucleotide polymorphism (SNP) detection (1b). In FIG. 1B, targets are amplified with a conventional primer matched with a primer with an Abortive Promoter Cassette (APC) at its 5′ end. Conversion of the APC from a single-stranded form to a duplex form activates it for Abscription. Addition of RNA polymerase along with a dinucleotide initiator and a single NTP allows production of the encoded trinucleotide Abscript RNA (2a). Inclusion of 3 NTPs allows synthesis of an 11 nucleotide (nt) Abscript (2b). Trinucleotide Abscripts are detected directly by LC-MS (3a). The 11 nt Abscripts are detected indirectly by Abscript-mediated molecular beacon opening (3b).

FIGS. 2A-2C illustrate Abscript detection by fluorescence. FIG. 2A is a diagram illustrating the steps involved in Abscript quantification based on their ability to participate in molecular beacon activation. The molecular beacon has a fluorescein (F) attached to the 5′ end of the terminal duplex inverted repeat. The quencher BHQ-1® (Q) is located at the 3′end. An 11 nt Abscript (dashed line) generated from an amplicon-associated APC anneals adjacent to the quencher. Primer extension of the Abscript by the CAP-DNA polymerase forces the beacon to the open configuration in which it is capable of fluorescence emission. FIG. 2B shows plots of fluorescence increase as a function of Abscription time for a set of calibrator DNAs. FIG. 2C shows a plot of calibrator concentration as a function of the calibrator rates of fluorescence increase. The resulting calibration formula is used to quantify sample DNA concentrations from fluorescence increase rates.

FIGS. 3A-3D illustrate MethylMagnet accuracy at low DNA inputs. In each of the figures, DNA inputs were bound in 2 μl reactions containing 0.1 μl of beads. Each complete fraction was amplified in a single PCR reaction to minimize sampling error for the methylation determination. FIGS. 3A and 3B give representative fluorescence increase per Abscription time for the HFE CpG island from normal cells and from artificially methylated HeLa DNA, respectively. FIGS. 3C and 3D show the results of CAP reactions for the SNRPN gene from DNA inputs of 6 genomic copies and 15 genomic copies. Quantitative results for the methylation analysis of HFE for normal DNA from saliva sediment verses artificially methylated HeLa and single measurements of SNRPN methylation at 6, 15 and 30 genomic copy inputs are shown in Table 1.

FIGS. 4A and 4B. Primers. The relationships among the 4 CAP primers is shown in the diagrams. The primer sequences are given in Table 2. FIG. 4A shows the primer locations during Phase 1. Forward and reverse target specific primers are linked at their 5′ ends to an APC-DN segment (forward primer, A-B) and a universal reverse segment (UR to the reverse primer C-D). The APC-DN segment encodes the downstream segment of a truncated inactive APC. When the target primers and appended segments are copied in Phase 1 of the PCR program, complements of the APC completion and the universal reverse primers are created. FIG. 4B shows the primer locations during Phase 2. In Phase 2 of the PCR program priming a B-D tagged amplicon with the APC completion primer (E) creates a full length functional APC. The universal reverse primer is designed to use the same PCR conditions as the APC completion primer. Use of the universal reverse segment simplifies primer design.

FIG. 5 is a map of the MGMT CpG island and 2 target fragments generated by AluI cleavage (Regions I and II). Methylation of either region I (37 CpGs) or II (28 CpGs) is sufficient to reduce MGMT expression. The gray segment in Region I (9 restriction sites) was analyzed by MSRE. Only Region I was analyzed by the molecular beacon and LC-MS methods in Table 4, below. Percent methylation scores are shown for molecular beacon detection verses LC-MS detection and MSRE. Methylation assignments are shown for the Epityper® method. The molecular beacon data was generated 3 years after the LC-MS experiment on the same DNA samples in Table 4. The molecular beacon and MSRE results were discordant for samples 15 19, 21 and 31. The MethylMeter results in these cases were consistent with survival data. The discordant result between the molecular beacon and LC-MS assignments for sample 15 is resolved in favor the molecular beacon method based on survival. Table 5 shows a comparison of the MethylMeter fluorescence assay data for recurrent glioma tumors for Regions I and II. MGMT mRNA data available for 3 discordant methylation assignments are consistent with the MethylMeter results.

FIGS. 6A-6D illustrate GliomaSTRAT results for low grade and high grade gliomas. FFPE samples were separated into methylated and unmethylated fractions followed by CAP reactions to measure the CIMP markers HFE, MAL, SOWAHA and the drug resistance marker MGMT., FIG. 6A is a map for SOWAHA. FIG. 6B is a map for MAL. FIG. 6C is a map for HFE. FIG. 6D is a map for IDH1. The maps show the locations of the targeted AluI fragments (unfilled rectangles), the MethylMeter PCR targets, (Black rectangles) and previously published MethyLight targets. The double arrow in HFE represents the CAP target for validating AluI cleavage. CAP reactions for the IDH1 R132H SNP were performed with a promoter-primer forming a mismatch at the site of the SNP. The results in Table 6 labeled Grade 2 indicate a CIMP-plus low grade glioma that is predicted to be responsive to Temozolomide. The results in Table 7 for sample labeled GBM is predicted to be a high grade glioma based on the CIMP-minus and IDH1 R132H minus results. The unmethylated status of MGMT predicts unresponsiveness to Temozolomide. The molecular results were consistent with a Pathologists' diagnoses.

FIGS. 7A and 7B illustrate the three primer system for abortive promoter assembly. FIG. 7A shows primers A, B and C. Primers A (arrow) and B (bold arrow) are forward and reverse target-specific primers respectively. Primer A has a truncated APC sequence at its 5′ end. This APC sequence includes an Abscript-encoding segment shown as a black rectangle. Oligonucleotide C (slashed arrow) encodes the upstream portion of the APC that is required to create a complete functional abortive promoter. The overlapping segments of primers A and C have identical sequence. FIG. 7B shows the Amplicons generated with the primers illustrated in FIG. 7A. The initial cycles of the PCR reaction involve primers A and B which create a duplex target with an incomplete APC (Amplicon I). Once the complement of APC-primer A is created in a duplex amplicon, primer C can anneal to that segment of the amplicon and create a longer amplicon with the complete APC (Amplicon II). Subsequent PCR cycles favor amplification by primer C over primer A based on differences in primer concentrations. The arrowheads in amplicons I and II align with the 3′ ends of the primers.

FIGS. 8A and 8B illustrate a four primer system for promoter assembly. FIG. 8A shows primers A, B, C and D. Primers A (arrow) and B (dotted arrow) are forward and reverse target-specific primers respectively. Primer A has a truncated APC sequence at its 5′ end. This APC sequence includes an Abscript encoding segment shown as a black rectangle. Primer B has a universal primer sequence at the 5′ end of its target-specific priming sequence. The complements of the truncated APC and the universal reverse primer sequences serve as priming sites for the APC completion primer C (slashed arrow) and the universal reverse primer D (bold arrow). FIG. 8B shows the Amplicons generated with the primers illustrated in FIG. 8A. Amplification of the target with primers A and B produces Amplicon I which has a truncated APC at one end and a universal reverse primer sequence at the other end. Amplification with primers C and D yield amplicon II containing the complete APC. Primers C and D are at a higher concentration than primers A and B to ensure efficient production of Amplicon II.

FIG. 9 is a graph showing the results of amplification of a segment of the MLH1 CpG island with a 4-primer APC assembly set. The indicated annealing temperatures were applied for the first 3 cycles to optimize the annealing of the target-specific primers (primers A and B, FIG. 2). Following PCR amplification the complete APCs were Abscribed to produce an 11-nt long Abscript that contributed to the opening of a fluorescein labeled molecular beacon. Signal strength is shown as the slope of the fluorescence increase per unit Abscription time.

### DETAILED DESCRIPTION

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. As used herein, the terms “comprises,” “comprising”, “includes”, and “including”, or any other variations thereof, are intended to cover a non-exclusive inclusion, such that a process, method, composition, reaction mixture, kit, or apparatus that comprises a list of elements does not include only those elements but may include other elements not expressly listed or inherent to such process, method, composition, reaction mixture, kit, or apparatus. The section headings used herein are for organizational purposes only and are not to be construed as limiting the subject matter described.

Unless specific definitions are provided, the nomenclatures utilized in connection with, and the laboratory procedures and techniques of molecular biology, biochemistry, and organic chemistry described herein are those known in the art. Standard chemical and biological symbols and abbreviations are used interchangeably with the full names represented by such symbols and abbreviations. Thus, for example, the terms “deoxyribonucleic acid” and “DNA” are understood to have identical meaning. Standard techniques may be used e.g., for chemical syntheses, chemical analyses, recombinant DNA methodology, and oligonucleotide synthesis. Reactions and purification techniques may be performed e.g., using kits according to manufacturer's specifications, as commonly accomplished in the art or as described herein. The foregoing techniques and procedures may be generally performed according to conventional methods well known in the art and as described in various general or more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)); Ausubel et al. Current Protocols in Molecular Biology (John Wiley & Sons Inc., N.Y. (2003)), the contents of which are incorporated by reference herein in their entirety for any purpose.

“About” as used herein means that a number referred to as “about” comprises the recited number plus or minus 1-10% of that recited number. For example, “about” 50 nucleotides can mean 45-55 nucleotides or as few as 49-51 nucleotides depending on the situation. Whenever it appears herein, a numerical range, such as “45-55”, refers to each integer in the given range; e.g., “45-55 nucleotides” means that the nucleic acid can contain 45 nucleotides, 46 nucleotides, etc., up to and including 55 nucleotides. Where a range described herein includes decimal values, such as “1.2% to 10.5%”, the range refers to each decimal value of the smallest increment indicated in the given range; e.g. “1.2% to 10.5%” means that the percentage can be 1.2%, 1.3%, 1.4%, 1.5%, etc. up to and including 10.5%; while “1.20% to 10.50%” means that the percentage can be 1.20%, 1.21%, 1.22%, 1.23%, etc. up to and including 10.50%.

“Transcription” as used herein, refers to the enzymatic synthesis of an RNA copy of one strand of DNA (i.e., template) catalyzed by an RNA polymerase (e.g. a DNA-dependent RNA polymerase).

“Abortive transcription” is an RNA polymerase-mediated process that reiteratively synthesizes and terminates the synthesis of oligonucleotides that correspond to at least one portion of a complementary nucleic acid template sequence. Abortive oligonucleotides synthesized in vivo vary in length of nucleotides, and are complementary to a sequence at or near the transcription initiation site.

“Abscription” is a form of abortive transcription optimized for in vitro analytical use to reiteratively produce short, uniform RNA transcripts or “Abscripts” from synthetic or naturally occurring promoter sequences at high frequency in vitro. The term “Abscripts” (capitalized), is used herein to distinguish optimized, synthetic transcripts produced in an in vitro Abscription reaction or assay, from the more general term “abscripts”, which also encompasses short abortive transcripts that are produced during the normal course of transcription as it occurs in nature.

“Reiterative” refers to the repetitive synthesis of multiple identical or substantially identical copies of a sequence of interest.

“Terminator” or “transcription terminator” as used herein, refers to an RNA chain terminating compound, complex or process. A terminator of the invention can, for example, be a nucleotide analog, which can be incorporated into an RNA chain during RNA synthesis to prevent the addition of additional nucleotides to the RNA chain.

“Amplification” as used herein, refers to the process of making identical copies of a polynucleotide, such as a DNA fragment or region. Amplification is generally accomplished by polymerase chain reaction (PCR), but other methods known in the art may be suitable to amplify DNA fragments of the invention.

A “target DNA sequence” or “target DNA” or “target” is a DNA sequence of interest for which detection, characterization or quantification is desired. The actual nucleotide sequence of the target DNA may be known or not known. Target DNAs are typically DNAs for which the CpG methylation status is interrogated. A “target DNA fragment” is a segment of DNA containing the target DNA sequence. Target DNA fragments can be produced by any method including e.g., shearing or sonication, but most typically are generated by digestion with one or more restriction endonucleases.

As used herein, a “template” is a polynucleotide from which a complementary oligo- or polynucleotide copy is synthesized.

“Synthesis” generally refers to the process of producing a nucleic acid, via chemical or enzymatic means. Chemical synthesis is typically used for producing single strands of a nucleic acid that can be used and primers and probes. Enzyme mediated “synthesis” encompasses both transcription and replication from a template. Synthesis includes making a single copy or multiple copies of the target. “Multiple copies” means at least 2 copies. A “copy” does not necessarily mean perfect sequence complementarity or identity with the template sequence. For example, copies can include nucleotide analogs, intentional sequence alterations (such as sequence alterations introduced through a primer comprising a sequence that is hybridizable, but not complementary, to the template), and/or sequence errors that occur during synthesis.

The terms “polynucleotide” and “nucleic acid (molecule)” are used interchangeably to refer to polymeric forms of nucleotides of any length. The polynucleotides may contain deoxyribonucleotides, ribonucleotides and/or their analogs. Nucleotides may be modified or unmodified and have any three-dimensional structure, and may perform any function, known or unknown. The term “polynucleotide” includes single-stranded, double-stranded and triple helical molecules. The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers.

“Oligonucleotide” refers to polynucleotides of between 2 and about 100 nucleotides of single- or double-stranded nucleic acid, typically DNA. Oligonucleotides are also known as oligomers or oligos and may be isolated from genes and other biological materials or chemically synthesized by methods known in the art. A “primer” refers to an oligonucleotide containing at least 6 nucleotides, usually single-stranded, that provides a 3′-hydroxyl end for the initiation of enzyme-mediated nucleic acid synthesis. A “polynucleotide probe” or “probe” is a polynucleotide that specifically hybridizes to a complementary polynucleotide sequence. As used herein, “specifically binds” or “specifically hybridizes” refers to the binding, duplexing, or hybridizing of a molecule to another molecule under the given conditions. Thus, a probe or primer “specifically hybridizes” only to its intended target polynucleotide under the given binding conditions, and an antibody “specifically binds” only to its intended target antigen under the given binding conditions. The given conditions are those indicated for binging or hybridization, and include buffer, ionic strength, temperature and other factors that are well within the knowledge of the skilled artisan. The skilled artisan will also be knowledgeable about conditions under which specific binding can be disrupted or dissociated, thus eluting or melting e.g, antibody-antigen, receptor-ligand and primer-target polynucleotide combinations.

“Nucleic acid sequence” refers to the sequence of nucleotide bases in an oligonucleotide or polynucleotide, such as DNA or RNA. For double-strand molecules, a single-strand may be used to represent both strands, the complementary stand being inferred by Watson-Crick base pairing.

The terms “complementary” or “complementarity” are used in reference to a first polynucleotide (which may be an oligonucleotide) which is in “antiparallel association” with a second polynucleotide (which also may be an oligonucleotide). As used herein, the term “antiparallel association” refers to the alignment of two polynucleotides such that individual nucleotides or bases of the two associated polynucleotides are paired substantially in accordance with Watson-Crick base-pairing rules. Complementarity may be “partial,” in which only some of the polynucleotides' bases are matched according to the base pairing rules. Or, there may be “complete” or “total” complementarity between the polynucleotides. Those skilled in the art of nucleic acid technology can determine duplex stability empirically by considering a number of variables, including, for example, the length of the first polynucleotide, which may be an oligonucleotide, the base composition and sequence of the first polynucleotide, and the ionic strength and incidence of mismatched base pairs.

As used herein, the term “hybridization” is used in reference to the base-pairing of complementary nucleic acids, including polynucleotides and oligonucleotides containing 6 or more nucleotides. Hybridization and the strength of hybridization (i.e., the strength of the association between the nucleic acids) is impacted by such factors as the degree of complementary between the nucleic acids, the stringency of the reaction conditions involved, the melting temperature (Tₘ) of the formed hybrid, and the G:C ratio within the duplex nucleic acid. Generally, “hybridization” methods involve annealing a complementary polynucleotide to a target nucleic acid (i.e., the sequence to be detected either by direct or indirect means). The ability of two polynucleotides and/or oligonucleotides containing complementary sequences to locate each other and anneal to one another through base pairing interactions is a well-recognized phenomenon.

A “complex” is an assembly of components. A complex may or may not be stable and may be directly or indirectly detected. For example, as described herein, given certain components of a reaction and the type of product(s) of the reaction, the existence of a complex can be inferred. For example, in the abortive transcription method described herein, a complex is generally an intermediate with respect to a final reiterative synthesis product, such as a final abortive transcription or replication product.

“Methylation” refers to the addition of a methyl group (—CH₃) to a molecule, typically to a nucleotide base in DNA or RNA. “mCpG” refers to a 5′-CG-3′ dinucleotide in which the C is methylated at position 5 (5-methylcytosine or 5-Me C). “CpG islands” are regions of genomic that contain a high frequency of the CpG dinucleotide. CpG Islands are in or near approximately 40% of promoters of mammalian genes and about 70% of human promoters have a high CpG content. See e.g. Fatemi et al. (2005) Nucleic Acids Res. 33:e176. doi:10.1093/nar/gni180. PMID 16314307.

“Promoter” as used herein, refers to a region of DNA that facilitates the transcription of an adjacent gene. Promoters are typically 5′ and proximal to the start site of transcription initiation in a gene, and direct an RNA polymerase and associated transcription factors to the correct location for transcription of the gene.

“Microarray” and “array,” are used interchangeably to refer to an arrangement of a collection of compounds, samples, or molecules such as oligo- or polynucleotides. Arrays are typically “addressable” such that individual members of the collection have a unique, identifiable position within the arrangement. Arrays can be formed on a solid substrate, such as a glass slide, or on a semi-solid substrate, such as nitrocellulose membrane, or in vessels, such as tubes or microtiter plate wells. A typical arrangement for an array is an 8 row by 12 column configuration, such as with a microtiter plate, however, other arrangements suitable for use in the methods of the present invention will be well within the knowledge of the skilled artisan.

The term “solid support” refers to any solid phase that can be used to immobilize e.g., a capture probe or other oligo- or polynucleotide, a polypeptide, an antibody or other desired molecule or complex. Suitable solid supports will be well known in the art and include, but are not limited to, the walls of wells of a reaction tray, such as a microtiter plate, the walls of test tubes, polystyrene beads, paramagnetic or non-magnetic beads, glass slides, nitrocellulose membranes, nylon membranes, and microparticles such as latex particles. Typical materials for solid supports include, but are not limited to, polyvinyl chloride (PVC), polystytrene, cellulose, agarose, dextran, glass, nylon, latex and derivatives thereof. Further, the solid support may be coated, derivatized or otherwise modified to promote adhesion of the desired molecules and/or to deter non-specific binding or other undesired interactions. The choice of a specific “solid phase” is usually not critical and can be selected by one skilled in the art depending on the methods and assays employed. Conveniently, the solid support can be selected to accommodate various detection methods. For example, 96 or 384 well plates can be used for assays that will be automated, for example by robotic workstations, and/or those that will be detected using, for example, a plate reader. For methods of the present invention that may involve e.g. an autoradiographic detection step utilizing a film-based visualization, the solid support may be a thin membrane, such as a nitrocellulose or nylon membrane, a gel or a thin layer chromatography plate. Suitable methods for immobilizing molecules on solid phases include ionic, hydrophobic, covalent interactions and the like, and combinations thereof. However, the method of immobilization is not typically important, and may involve uncharacterized adsorbtion mechanisms. A “solid support” as used herein, may thus refer to any material which is insoluble, or can be made insoluble by a subsequent reaction. The solid support can be chosen for its intrinsic ability to attract and immobilize a capture reagent. Alternatively, the solid support can retain additional molecules which have the ability to attract and immobilize e.g., a “capture” reagent.

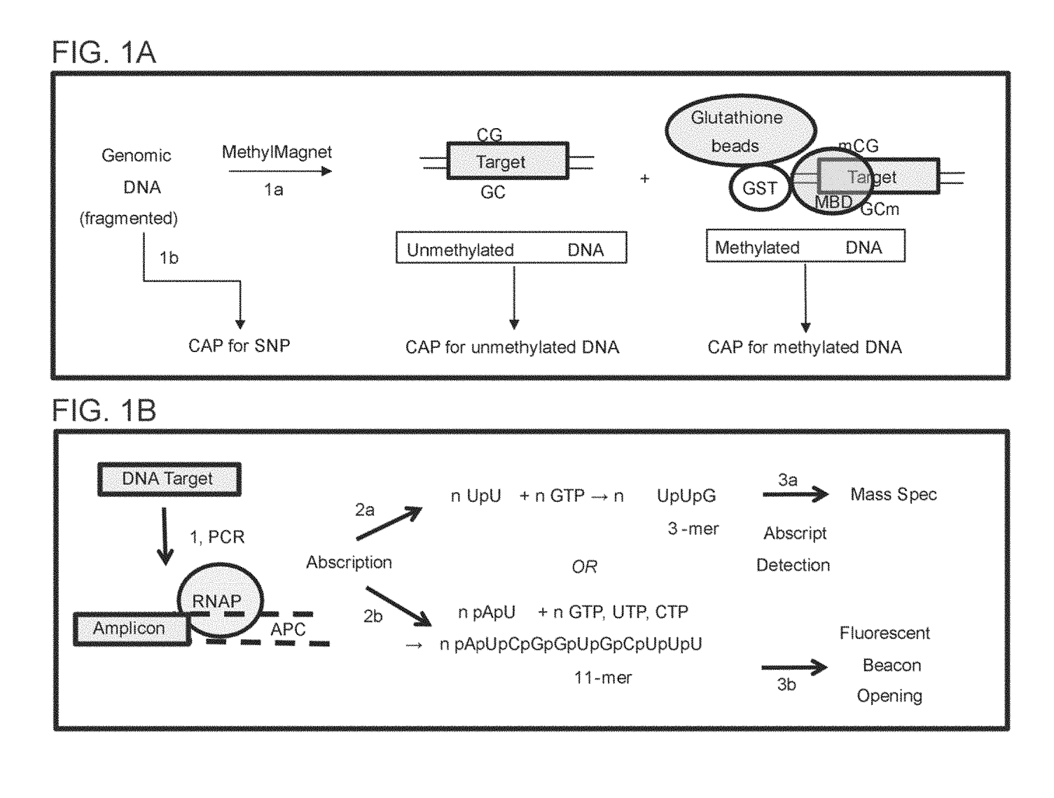

The present invention is based on a molecular detection technology called Abscription which is in turn based on the natural phenomenon known as abortive transcription. See e.g., U.S. Pat. No. 8,263,339 at FIG. 1. Abscription is a robust, isothermal method for detecting and quantifying a wide range of targets including proteins, nucleic acids, SNPs and CpG methylation. U.S. Pat. Nos. 7,045,319; 7,226,738; 7,468,261; 7,470,511; 7,473,775; 7,541,165; 8,263,339; 8,211,644, 8,242,243; and 8,211,644). Abscription occurs during the initiation phase of transcription in which RNA polymerase (RNAP) reiteratively generates short RNAs, or aborted transcripts (Abscripts), while remaining tightly bound to the promoter (Hsu, Biochim. Biophys. Acta (2002) 1577:191-207; Hsu et al. Biochemistry (2003) 42:3777-86; Vo et al. Biochemistry (2003) 42:3798-811; Vo et al. Biochemistry (2003) 42:3787-97; Hsu et al. Biochemistry (2006) 45:8841-54). The sequences of the promoter and the initially transcribed segment have significant effects on the lengths of the predominant Abscripts, as well as their rates of synthesis (Hsu et al. Biochemistry (2006) 45:8841-54.28). Multiple optimal highly abortive promoters, called Abortive Promoter Cassettes (APCs), have been developed and optimized to make Abscripts of different sequences and lengths (between 3 and 12 nt) at extremely high rates.

The generation of short Abscripts is very efficient because the RNAP does not dissociate from the promoter between rounds of truncated RNA synthesis, as it does after producing each full length transcript, and will continue to produce Abscripts at high turnover rates until substrates are depleted. This results in the very rapid production of thousands of Abscripts per APC each minute. Abscription is a signal amplification, rather than a target amplification process.

“Molecular beacons” or “beacons” are single-stranded oligonucleotide hybridization probes that form a stem-and-loop structure. The loop contains a probe sequence that is complementary to a nucleic acid sequence, and the stem is formed by the annealing of complementary “arm” sequences that are located on either side of the probe sequence. Exemplary molecular beacons are described in U.S. Pat. No. 8,211,644 (see e.g., FIGS. 10A-15B). The skilled artisan will recognize that many additional molecular beacon sequences are commercially available and additional molecular beacon sequences can be designed for use in the methods of the present invention. A detailed discussion of the criteria for designing effective molecular beacon nucleotide sequences can be found on the world wide web at molecular-beacons (dot) org/PA_design (dot) html, and in Marras et al. (2003) “Genotyping single nucleotide polymorphisms with molecular beacons.” (In Kwok, P. Y. (ed.), Single nucleotide polymorphisms: methods and protocols. The Humana Press Inc., Totowa, N.J., Vol. 212, pp. 111-128); and Vet et al. (2004) “Design and optimization of molecular beacon real-time polymerase chain reaction assays.” (In Herdewijn, P. (ed.), Oligonucleotide synthesis: Methods and Applications. Humana Press, Totowa, N.J., Vol. 288, pp. 273-290), the contents of which are incorporated herein by reference in their entirety. Molecular beacons can also be designed using dedicated software, such as called ‘Beacon Designer,’ which is available from Premier Biosoft International (Palo Alto, Calif.), the contents of which is incorporated herein by reference in its entirety.

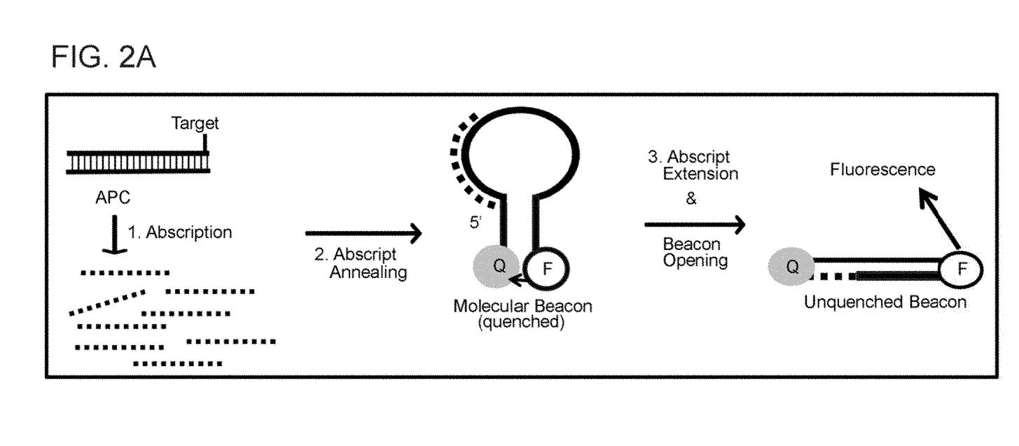

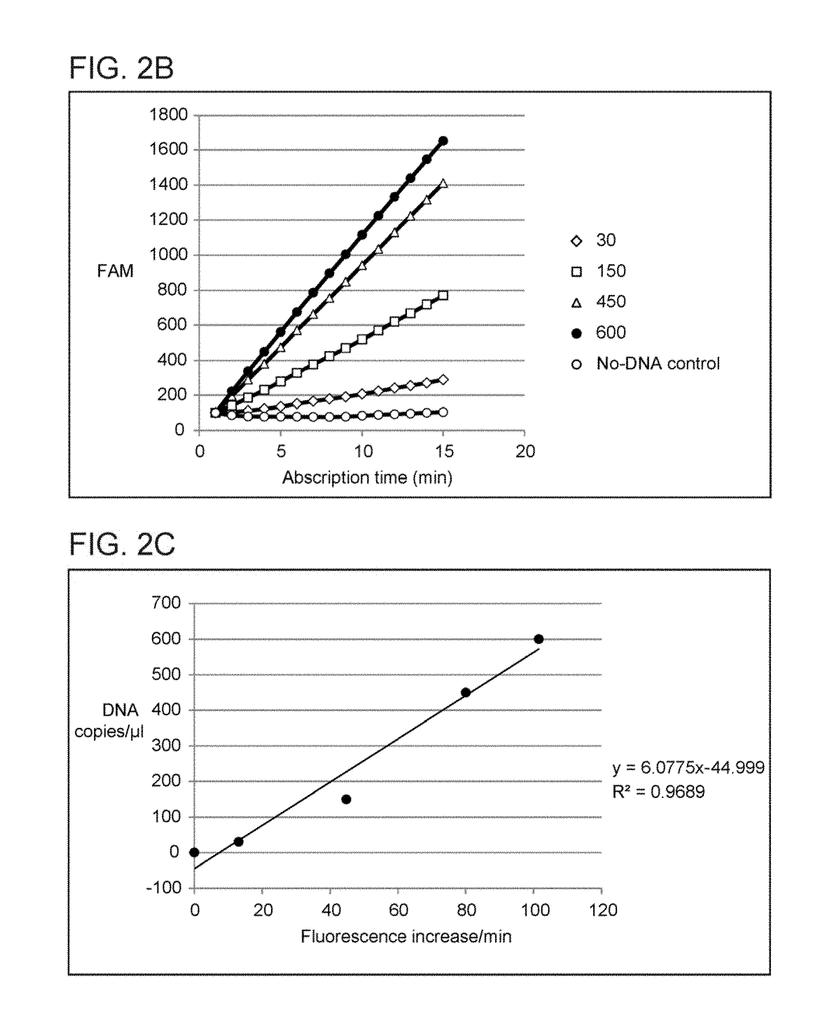

A fluorophore is covalently linked to the end of one arm of the molecular beacon sequence and a fluorescence quencher is covalently linked to the end of the other arm. Molecular beacons do not fluoresce when they are free in solution under suitable conditions of temperature and ionic strength (e.g. below the Tₘ of the stem-loop structure). However, when molecular beacons hybridize to a nucleic acid complementary to the molecular beacon probe region, they undergo a conformational change that enables them to fluoresce brightly. In the absence of a complementary nucleic acid, the probe is dark, because the stem places the fluorophore so close to the fluorescence quencher that the fluorophore and quencher transiently share electrons, eliminating the ability of the fluorophore to emit fluoresce. When the probe encounters a suitable complementary nucleic acid molecule, it forms a probe-target hybrid that is longer and more stable than the stem hybrid. The rigidity and length of the probe-target hybrid precludes the simultaneous existence of the stem hybrid. Consequently, the molecular beacon undergoes a spontaneous conformational reorganization that forces the stem hybrid to dissociate and the fluorophore and the quencher to move away from each other, thereby allowing the fluorphore to emit fluorescence upon excitation with a suitable light source. See e.g. U.S. Pat. No. 8,211,644 at FIG. 2.

Abscript reactions are isothermal and do not require cycles that include high temperature denaturation. Because unopened molecular beacons are dark, it is not necessary to isolate opened beacon to measure the signal in an assay. Thus, beacon-based Abscription can be performed in real-time by including molecular beacons into Abscription target detection reactions.

The present invention provides DNA methylation detection techniques that are bisulfite-free, sensitive, quantitative and suitable for use with FFPE and other clinical samples. The methods described herein combine affinity fractionation of DNA with Coupled Abscription PCR (CAP). The methods include a fluorescence readout that is based on Abscript-dependent opening of a molecular beacon. The methods of the invention are sensitive enough for quantitative, multi-target DNA methylation profiling of clinical samples.

“MethylMeter”, as used herein, refers to a method for detecting DNA methylation comprising affinity separation of DNA into methylated and unmethylated fractions followed by CAP. In certain aspect of the invention, CAP quantitatively determines the amount of DNA methylation in a sample of genomic DNA. In other aspects of the invention, CAP quantitatively determines the amount of DNA methylation in a specific sequence present in the sample, particularly methylation of CpG islands.

The MethylMeter methods of the invention can accurately measure methylation with DNA inputs as small as 6 genomic copies. The molecular beacon read-out is consistent with LC-MS readout sensitivity and accuracy. The MethylMeter molecular beacon analysis of the present invention of MGMT methylation is consistent with patient survival and mRNA data.

Furthermore, coupled Abscription PCR methods of the invention can be can be used for SNP detection on FFPE samples.

The present invention provides two improved versions of the MethylMeter assay and its application to the analysis of clinical samples. In Format I, the Abscript produced is an 11-mer that initiates the opening of a fluorescent molecular beacon and can be measured on a qPCR or other fluorescent instrument. Tyagi & Kramer, Nat Biotechnol. 14: 303-8 (1996). In Format II, the Abscript produced is a trinucleotide that is detected by mass spectrometry. This method has high multiplexing potential, but requires access to a mass spectrometer not available in many laboratories.

Thus, the present invention provides methods for detecting CpG island methylation by first separating methylated DNA comprising at least one CpG island from unmethylated DNA in a sample and then performing Coupled Abscription PCR to detect the presence of the at least one CpG island nucleotide sequence in the methylated DNA, thereby detecting CpG island methylation. Typically, the methylated DNA is genomic DNA, such as human genomic DNA. Separation of methylated DNA can be accomplished using any affinity reagent that specifically binds methylated CpG dinucleotides. In certain embodiments, the affinity reagent is an MBD protein or domain, such as Methyl-CpG-Binding Protein 2 (MBD2), which can be the GST-MBD GST-MBD2 fusion protein described in U.S. Pat. Nos. 8,211,644 and 8,242,243, the contents of which is incorporated by reference herein in their entirety.

Coupled Abscription PCR can be used to qualitatively or quantitatively detect the presence of the at least one CpG island nucleotide sequence. In certain embodiments of the invention, CAP uses three primers: a forward target-specific primer that has a truncated Abortive Promoter Cassette (APC) sequence at its 5′ end; a reverse target-specific primer for creating an amplicon that contains a duplex inactive APC; and an APC primer that overlaps with the 5′ end of the truncated APC sequence. Amplifying the target with the three primers fuses an APC to the target sequence and when used in the CAP reaction (including RNAP), Abscripts are produced that can be detected by Mass Spectrometry or by fluorescence (utilizing molecular beacons).

In other embodiments of the invention, CAP uses four primers: a forward target-specific primer that has a truncated APC sequence at its 5′ end; a reverse target-specific primer comprising a universal primer sequence at 5′ end of a target-specific priming sequence; an APC completion primer that overlaps with the 5′ end of the truncated APC sequence; and a universal reverse primer. Amplifying the target with the four primers fuses an APC to the target sequence and when used in the CAP reaction (including RNAP), Abscripts are produced that can be detected by Mass Spectrometry or by fluorescence (utilizing molecular beacons).

In certain aspects of the invention, multiple targets are detected, such as two or more target regions of a single gene that contain CpG islands, or two or more target genes that contain CpG islands. The targets can be analyzed in an array format, such as in a multi-well plate and where fluorescent PCR products are detected, can use standard Real Time PCR equipment.

The assay was validated by measuring the DNA methylation of two regions of the MGMT promoter CpG island and the CpG islands associated with three genes that determine the Glioma CpG Island Methylator Phenotype (G-CIMP). Noushmehr et al., Cancer Cell. 17:510-22 (2010). Results for MGMT methylation have been compared to those generated using bisulfate-free MSRE or bisulfate-based, mass-spec based Epityper® on RCL2 fixed samples from the same tumor. The original eight gene G-CIMP panel was reduced three genes with 100% agreement with the eight gene profile. See Noushmehr et al. Cancer Cell. 17:510-22 (2010); (data not shown). The G-CIMP result was further validated with an Abscription-based assay on the same samples for the IDH1 R132H SNP that has been shown to cause CIMP. Turcan et al., Nature 483:479-83 (2012). It has become common practice to determine the IDH1 and IDH2 mutational status to infer CIMP because existing methods for DNA methylation detection lack the sensitivity to reliably measure the methylation of a multi-gene panel in FFPE samples.

The methods of the present invention have the ability to simultaneously grade gliomas by determining CIMP with methylation level of three genes and determine the tumor's response to first line chemotherapy using MGMT methylation. The assays have been used to verify the CIMP status by an additional CAP based test that detects the major SNP believed to cause CIMP, the IDH1 R132H mutation. The assay simultaneously measures the WT allele and a ratio of mutation to WT is generated.

A major advantage of the MethylMeter assays of the present invention is their sensitivity and ability to detect even small DNA targets (≧40 bp) in the size range typical of DNA found in many clinical samples, including FFPE, blood and urine. When used in the correct proportion, the GST-MBD2 magnetic beads are able to accurately fractionate methylated DNAs over the DNA input range of 6 genomic copies to 948,000 copies. The affinity approach avoids chemical damage to the sample that occurs with bisulfate-based methods. The addition of the Abscription-based linear signal amplification to the PCR target amplification increases the sensitivity of the assay beyond that achieved with qPCR. See McCarthy et al. (2012), supra. Consequently a high success rate with FFPE samples is possible without pre-amplification or nested PCR. Unlike in qPCR, every amplicon generates multiple signals. qPCR has the advantage of a one-tube homogeneous format. Because the Abscription step produces linear signal amplification it is not necessary to take precautions against cross contamination among PCR reactions during the Abscription set-up.

The MethylMeter methods of the invention are highly accurate, based on their consistency with survival data and MGMT mRNA levels in cases of discordance with other methylation detection methods. Affinity methods have the advantage that they are less sensitive to methylation pattern heterogeneity than methods that target a few specific CpG sites. For example, the presence of one or more unmethylated CpG sites in a targeted site of an otherwise methylated DNA can lead to an incorrect inference of the overall methylation status for methods based on MS-PCR or MRSE. Yegnasubramanian et al., Nucleic Acids Res. 34:e19 (2006). This situation should not affect affinity-based detection because every methylated CpG site can contribute to a binding event.

MethylMeter assay development, as disclosed herein, is simple compared to other methods because the discrimination between methylated and unmethylated DNAs is performed in a standardized binding reaction. Most gene targets can be analyzed using the same binding conditions, although the stringency of the binding reaction can be changed by optimizing the NaCl concentration of Wash Buffer 2. Primer design in MethylMeter has fewer constraints than with bisulfite-based or MSRE methods. Target sequence complexity is not reduced by bisulfite treatment so more potential priming sites are available. Target specific primers can be placed at any arbitrary region in a target sequence. There is no requirement to include or exclude CpG sites. Forward and reverse primers can be placed arbitrarily close, as there are no probes to accommodate. This is an advantage in development of assays for heavily damaged sample DNAs or for cell-free DNA from bodily fluids. Amplification targets as small as 40 nt have been developed. A further simplification is that the Abscription signal is independent of the target sequence, so Abscription will proceed with equal efficiency with any gene target that is tagged with the same APC.

The nondestructive nature of MethylMagnet allows the DNA in leftover methylated or unmethylated fractions to be used for other assays. This flexibility can be useful if sample sizes are severely constrained. The knowledge that the IDH1 R132 SNP is not associated with a CpG island has been exploited to perform IDH1 SNP detection from the unmethylated MethylMagnet fraction. Thus, in certain aspects, a Single Nucleotide Polymorphism (SNP) is also detected by methods of the invention.

In certain aspects of the invention, methods are provided to assemble and fuse a full length Abortive Promoter Cassette (APC) to a target nucleic acid during PCR amplification of the target. The linked APC is used to quantify amplicon abundance by the production of RNA Abscripts from the synthetic APC. Stepwise PCR-dependent promoter assembly allows for target-fusion of APCs that are too long to be synthesized as monolithic promoter-primer oligonucleotide reagents.

Coupled Abscription PCR (CAP) increases the sensitivity of PCR by adding a signal amplification step to the target amplification associated with PCR. APCs are fused to target sequences through the use of target-specific primers linked to inactive single-stranded APCs. Target DNA sequences are detected by Abscription when the APC is used to synthesize many copies of a short abortive RNA transcript (Abscript). The Abscript is an arbitrary sequence encoded by the APC. Different Abscript detection methods require APCs of different lengths. Detection methods for short Abscripts (trinucleotides) require APCs that are sufficiently short that their full length versions can be accommodated in promoter-primers. Detection based on the opening of molecular beacons via Abscripts requires APCs that are greater than 80 nt in length. The combination of the APC and target-specific primer sequence is too long to allow economical production of such a promoter-primer reagent.

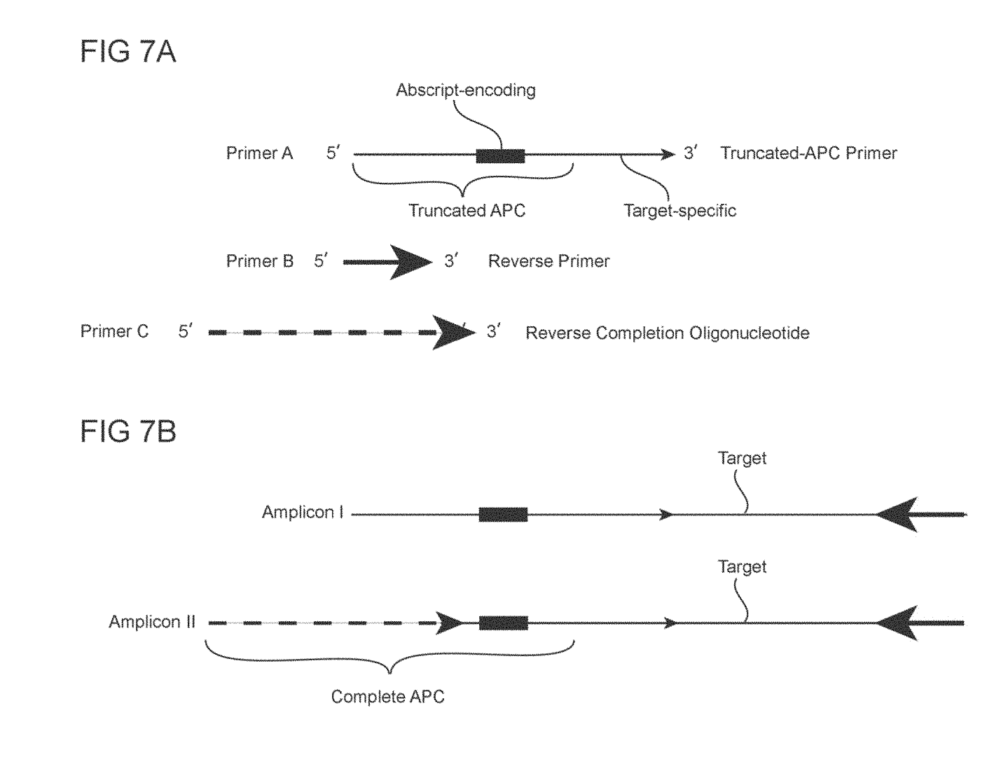

FIGS. 7A and 7B shows the strategy for assembling long APCs onto target strands using 3 primers. A forward target-specific primer has a truncated APC sequence at its 5′ end (FIG. 7A). The APC sequence encodes the Abscript but lacks upstream APC sequence that is necessary for activity. Primer A is used with reverse target-specific primer B to create amplicon I which contains a duplex inactive APC. APC primer C overlaps with the 5′ end of the truncated APC sequence in primer A. The overlapping sequence is identical in both primers thereby minimizing direct interactions between them. Creation of amplicon I allows primer C to participate in amplification of the amplicons in cooperation with primer B. Primer A is set to a low concentration relative to primers B and C to minimize competition between primers A and C for priming amplicons.

A limitation of the 3 primer system in FIGS. 7A and 7B is that the APC completion primer (primer C) must have a melting temperature (Tₘ) that is compatible with the target-specific reverse primer. To avoid the necessity of designing different APC completion primers for different targets, a 4 primer system according to one embodiment of the invention incorporates a universal reverse priming sequence that is compatible with the APC completion primer.

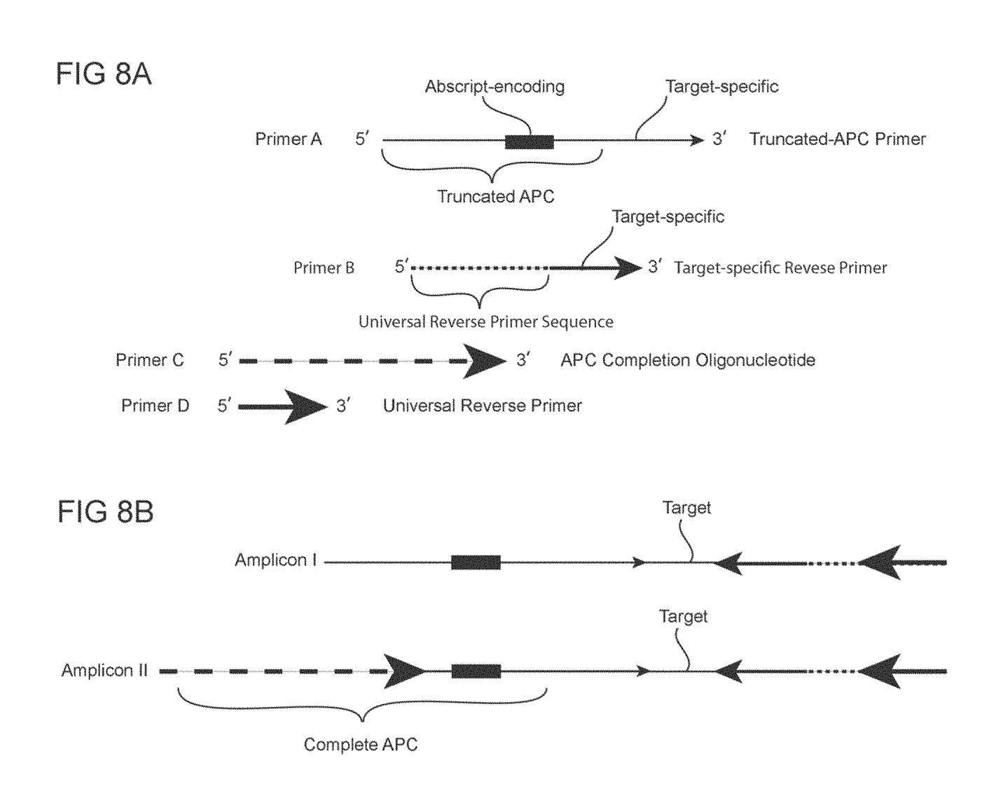

FIGS. 8A and 8B show the components of the 4 primer system. Primer B is modified with an arbitrary universal sequence that has the same Tₘ as the APC-completion primer (primer C). The target is copied with primers A and B to create an amplicon with a partial APC at one end and a universal primer sequence at the other end. Once the complements of the APC and the universal primer sequences are created further amplification can occur with primer C and the universal reverse primer (primer D). The same amplicon amplification condition can be used with any target in this system because primers C and D are optimized to work under the same PCR conditions. These primers are present at a higher concentration than the target-specific primers to heavily bias amplification to produce amplicons with full-length APCs.

FIGS. 8A and 8B illustrate a four primer system for promoter assembly. Primers A and B are forward and reverse target-specific primers respectively. Primer A has a truncated APC sequence at its 5′ end. This APC sequence includes an Abscript encoding segment shown as a black rectangle. Primer B has a universal primer sequence at the 5′ end of its target-specific priming sequence. Amplification of the target with primers A and B produces amplicon I which has a truncated APC at one end and a universal reverse primer sequence at the other end. The complements of the truncated APC and the universal reverse primer sequences serve as priming sites for the APC completion primer C and the universal reverse primer D. Amplification with primers C and D yield amplicon II containing the complete APC. Primers C and D are at a higher concentration than primers A and B to ensure efficient production of amplicon II.

The present invention also provides kits for performing the methods of the invention which can include one or more of Abscriptase, control templates, methylation affinity reagents (e.g. MethylMagnet GST-MBD2 fusion protein) and/or one or more primers as described herein. In certain embodiments, the primers include one or more primers selected from the group consisting of SEQ ID NOs:1-38. The kits of the invention may contain virtually any combination of the components set out above or described elsewhere herein. As one skilled in the art would recognize, the components supplied with kits of the invention will vary with the intended use for the kits. Thus, kits may be designed to perform various functions set out in this application and the components of such kits will vary accordingly.

Throughout this application, various publications are referenced. The disclosures of all of these publications and those references cited within those publications in their entireties are hereby incorporated by reference into this application in their entirety in order to more fully describe the state of the art to which this invention pertains.

The invention will now be described with reference to the following EXAMPLES, which are provided to further explain but in no way limit the scope of the invention.

### EXAMPLES

### Example 1

### Amplification of the MLH1 CpG Island with a 4-Primer Set That Encodes an 11 nt Abscript

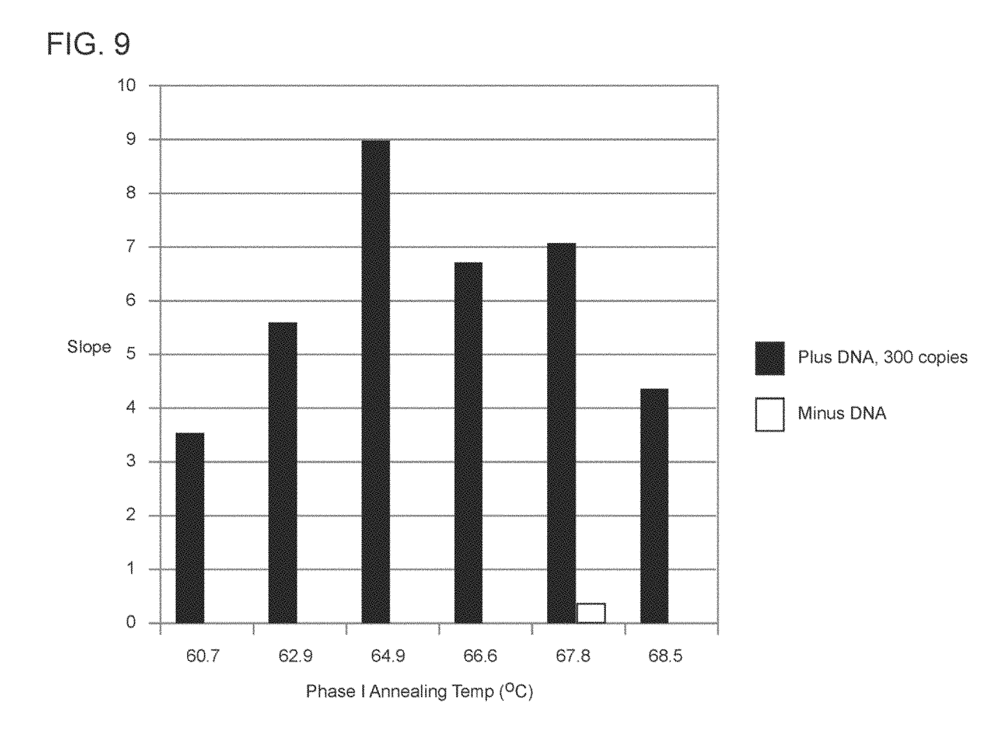

A segment of the MLH1 CpG island was amplified with a set of 4 primers that were designed as depicted in FIGS. 8A and 8B. HeLa genomic DNA (150 copies) was subjected to hot start PCR followed by Abscription under conditions where the Abscript contributes to the opening of a molecular beacon (FIG. 9). The first 3 PCR cycles were performed at 6 annealing temperatures to determine the most optimal conditions for the target specific primer sequences (FIG. 9, Legend). The last 27 cycles used an annealing temperature of 57.7° C. which was optimal for the APC completion primer and the universal reverse primer (FIGS. 8 A and 8B, primers C and D). Following target amplification, the PCR reactions were supplemented with Abscription reagents and were incubated at 40° C. for 20 min to synthesize an 11-nt long Abscript. FIG. 9 shows the signal as the slopes of fluorescence intensity verses Abscription time due to the Abscript-dependent opening of the fluorescein-labeled molecular beacon. The most rapid signal generation occurred at the 64.9° C. with no background signal in the absence of DNA.

### Example 2

### Methylation Detection in FFPE Samples

### Materials & Methods

### Tissue & DNA Samples

Formalin Fixed Paraffin Embedded (FFPE) tissue sections from primary glioblastomas were obtained from the Austrian Institute of Technology (Vienna, Austria) and NuvOx Pharma (AZ, USA) and included tumors that had been assigned Grade II, III or IV via histopathology. Samples had been stored after fixation for between two months and 26 years before analysis. Tumor DNAs from frozen recurrent GBMs were provided by Tocagen Inc. (CA, USA).

### DNA Purification From FFPE Tumor Sections

FFPE tissue sections were deparaffinized in xylene in two 5 min incubations. Solvent changes were made by centrifugation at 14,000×g for 5 min. Residual xylene was removed with a 100% ethanol wash. The tissue pellets were air dried and suspended in 50 mM Tris-HCl (pH 8.5 at 10 mM and 25° C.), 100 mM NaCl, 1 mM EDTA, 0.5% v/v Tween-20, 0.5% w/v NP40, 20 mM DTT and 500 μg/ml Proteinase K. The samples were incubated overnight at 55° C. followed by a 20 min incubation at 80° C. to inactivate the Proteinase K and to reverse crosslinks. Nucleic acids were precipitated with 0.5 volume of 7.5 M ammonium acetate and 3 volumes of 100% ethanol. The pellets were dissolved in 50 μl of TE buffer.

### DNA Fragmentation

DNA was digested with 10 units of AluI in 20 mM Tris-HCl pH 8, 50 mM KCl, 2.8 mM MgCl₂ for 1 hour to overnight. The extent of cleavage was determined by Coupled Abscription-PCR (CAP) as described below. Each cleaved DNA sample and an uncut portion of each reaction was amplified with a primer pair that is sensitive to AluI cleavage due to an AluI site located between the priming sites in the HFE CpG island (forward primer target-specific sequence: 5′-AGGCACTCCCTCACGGGGTC; reverse primer target-specific sequence: 5′-GAGGGCTGCGGGCGAACTAG).

### Copy Number and Amplifiable DNA Determination

The number of genomic copies/μl was determined for the cut and uncut samples from a titration of uncut HeLa DNA that was amplified in parallel with the samples. The fraction of cleaved AluI test sites was expressed as 1-(DNA digest signal/uncut control signal). The uncut DNA concentration was taken as the amplifiable DNA content of each sample. The A₂₆₀ was used to calculate the total nucleic acid concentration of each sample. AluI digests were frozen at −20° C. without purification.

### Methylated DNA Fractionation

DNA samples were fractionated using the MethylMagnet® kit (RiboMed Biotechnologies, Calif., USA) following the standard protocol except that a 10× binding buffer was used to minimize the dilution of the samples. AluI-cut DNA was mixed with 2 μl of 10× Binding buffer (100 mM Tris-HCl pH 7.6 [at 10 mM and 25° C.], 1.6 M NaCl, 1 mM EDTA, 0.8% v/v TritonX-100, 40% v/v glycerol) and a variable amount of DNA dilution buffer (20 mM Tris-HCl pH 8, 50 mM KCl, 2.8 mM MgCl₂) to give a 20 μl binding reaction. The MgCl₂ component (final concentration 2.5 mM) was provided by the DNA sample and the dilution buffer. Amplifiable FFPE DNA inputs ranged from 3 ng to 20 ng.

Magnetic beads bearing a MethylMagnet GST-MBD2 fusion protein were distributed to 1.7 ml microcentrifuge tubes and were washed with 100 μl of Wash Buffer 1 without prior removal of the bead storage buffer. The beads were suspended in 20 μl DNA samples in 1× binding buffer and were incubated for 1 hr at room temperature (22° C.) at 1000 rpm in a thermomixer (Eppendorf, N.Y., USA). At the end of the binding reaction the beads were pelleted with a magnet and the supernatant fractions containing the unmethylated DNA were recovered. The bead pellets were washed twice with 400 μl of Wash Buffer 2 for 5 min at room temperature (22° C.) and at 1000 rpm in a thermomixer. The stringency of Wash Buffer 2 was 300 mM NaCl. A third 400 μl wash with TE buffer (10 mM Tris-HCl, pH 8, 1 mM EDTA) was performed without incubation. The bead pellets were suspended in 20 μl of Bead-Fraction buffer.

Bead-Fraction buffer had the same composition as one part 10× binding buffer to nine parts DNA dilution buffer to avoid possible PCR bias based on buffer differences between the methylated and unmethylated fractions. Methylated fractions from FFPE samples were stored as bead suspensions without eluting the DNA from the beads. Methylated DNA from frozen tissue was eluted from the beads by a 10 min incubation at 65° C. and 1000 rpm in a thermomixer. The beads were removed with a magnet. Eluted methylated DNA and unmethylated DNA fractions from frozen tissues were stored at −20° C. The fractions from FFPE samples were stored refrigerated for up to 3 months before CAP.

For higher throughput, samples were fractionated in polypropylene round bottom microtiter plates with reduced wash buffer volumes. All bead washes were 150 Sample mixing in microtiter plates was done at 850 rpm in a thermomixer.

### Methylated DNA Fraction of Samples With 30 Genomic Copies or Less

The accuracy of DNA fractionation by the MethylMagnet magnetic beads at extremely low DNA inputs was demonstrated with reduced binding reaction volume and reduced bead input.

Stock MBD-magnetic beads were diluted 100-fold into 1× Binding buffer and 10 μl were distributed to 0.5 ml microcentrifuge tubes. The beads were pulled-down with a magnet and the Binding buffer was discarded. The beads were suspended in 2 μl of DNA that had been diluted into 1× Binding buffer. The samples consisted of undamaged cell-pellet DNA from saliva, HeLa DNA and artificially methylated HeLa DNA. The DNA binding mixtures were incubated at 22° C., 1000 rpm for 1 hr. At the completion of the binding reaction, the beads were pulled down with a magnet and the 2 μl supernatant fractions containing the unmethylated DNAs were transferred to PCR tubes. The beads were washed with 18 μl of complete PCR master mix (including Taq enzyme) and the wash buffer (PCR master mix) was added to each unmethylated DNA fraction to create a complete PCR reaction mix. The beads which contained the methylated DNA fraction were suspended in 18 μl of complete PCR master mix and 2 μl of Bead fraction buffer. The methylated DNAs were amplified directly from the beads, similarly to the treatment of FFPE methylated fractions. Both fractions were processed by CAP Format I as described below, with the exceptions that end point PCR was done for 36 cycles and the Abscription reaction volume was 40 μl.

### CAP Format I

### Fluorescent Beacon Based Detection

The amount of target DNA in the methylated and unmethylated fractions is determined by performing CAP on each. When working with FFPE DNA or fewer than 30 copies of DNA from other sources, the methylated DNA was not eluted from the magnetic beads. DNA was measured by amplifying 1 μl samples of the unmethylated fraction and 1.08 μl of the suspended, bead-containing methylated fractions in 10 μl PCR reactions. Both the unmethylated and methylated DNA inputs from frozen tissues were 1 μl. The difference in the sample input volumes for FFPE DNA takes into account the volume occupied by the magnetic beads in the methylated fraction. The PCR master mix contained 0.5 units of Maxima Taq (ThermoFisher, Md., USA), 1× Maxima Hot start buffer, 0.8 mM dNTPs, 1.75 mM MgCl₂, 5.26% v/v DMSO and 1× target specific primers. Additional MgCl₂ was contributed by the DNA sample to give a final concentration of 2 mM. Additional DMSO was contributed by the primers to give a final concentration of 6% v/v. DMSO inclusion is essential for maximal amplification efficiency of heavily methylated fragments. Kholod et al., J Mol Diagn. 9:574-81 (2007); Pulverer et al., Clin Neuropathol. 33:50-60 (2014).

The PCR cycling conditions were: step 1, 95° C., 2.25 min; step 2, 57° C., 3 min; step 3, 72° C., 30 sec; step 4, repeat steps 1-3, 2×; step 5, 88.9° C., 5 sec; step 6, 57.7° C., 15 sec; step 7, 72° C., 15 sec; step 8, repeat steps 5-7 25× for 29 cycles or 27× for 31 cycles. Amplifiable DNA inputs to PCR reactions below 100 genomic copies were amplified for 31 cycles. Amplifiable DNA inputs to PCR reactions between 100 and 500 genomic copies were amplified for 29 cycles.

After PCR cycling the magnetic beads were removed using a magnetic rack designed for PCR tubes (Ribomed). The PCR reactions were combined with 10 μl of 1× Abscription mix containing pApU (20 mM), GTP, CTP, UTP (8 mM each), dATP, dGTP (8 mM each), 1× Abscriptase-I, 1× CAP-DNA polymerase and 1 ρM FAM-BHQ-1®-labeled molecular beacon. The Abscription reactions need not be carried out in a thermocycler. The Abscription phase is isothermal. However, it is convenient to carry out the entire reaction in a qPCR machine. Samples were incubated in an ABI 7000 Prism qPCR cycler for 15 repetitions of 40° C. for 6 sec and 41° C. for 50 sec. Each cycle was 1 min in duration when factoring in the 4 sec ramp time. Temperature cycling during the Abscription phase is not required and was only performed because of programming requirements on the machine.

### Data Analysis Format I

### Fluorescent Beacon Based Detection

The rate of Abscript-dependent fluorescence increase is directly proportional to the amount of amplifiable target DNA in the CAP reaction. For each batch of samples plus a no DNA control, a component file containing the FAM fluorescence measurements in row orientation was exported from the ABI 7000 to Microsoft Excel where the slopes of the FAM fluorescence increase verses Abscription time were calculated. Cycle number or min of Abscription can be used interchangeably because each cycle is 1 min long. First a row of Abscription minutes (cycle numbers) was set up in the Excel worksheet corresponding to all of the fluorescence readings. The slope was calculated using the Excel formula=SLOPE(FAM readings, Abscription minutes). The FAM and time ranges in the slope formula can be set to an arbitrary subset of the data to cover the linear portion of the relationship. The FAM increase rate for the no DNA control was subtracted from each of the sample FAM increase rates.

It was useful to plot the FAM increase verses Abscription time in order to confirm that the FAM increase was linear and to determine the range of the linearity. On occasion there is a lag before the linear accumulation of fluorescence. FAM values for all samples were normalized to a time=1 min value of 100. Graphs of FAM increase verses Abscription time were plotted using the Excel scatter plot option choosing the entire Abscription time range for the X-values and the entire FAM reading range for the Y-values (FIG. 2B).

The linear relationship between the FAM increase rate and the DNA PCR input was determined with a titration of HeLa DNA that was processed by CAP in parallel with the samples. The FAM increase rates for the calibrators were plotted using the Excel scatter plot option with FAM increase rates as the X-values and the calibrator DNA amounts as the Y-values (FIG. 2C). The experimental sample DNA amounts (y) were calculated from the sample rates of FAM increase (x), the slope (m) and intercept (b) of the calibration curve (y=mx+b).

The percent methylation was calculated as (Me/Me+U)*100, where Me is the amount of target in the methylated fraction and U is the amount of target in the unmethylated fraction.

### CAP Format II

### Mass Spec Based Detection

PCR reactions for LC-MS detection were done in 20 μl volumes containing 1× Maxima Taq Hot Start Buffer, 2 mM MgCl₂, 0.8 mM dNTPs, 6% v/v DMSO, PCR primers at 0.5 μM each, and 0.5 unit of hot start Maxima Taq (Fermentas, Md, USA). Unmethylated DNA fractions (2 μl) and suspended bead fractions containing methylated DNA (2.08 μl) were added to 18 μl of PCR master mix. The increased volume of the bead fraction compensated for the volume occupied by the suspended beads.

The cycling conditions were 3 cycles of 94° C. for 2 min, 65° C. (MGMT region I) or 67° C. (MGMT region II) for 30 sec, 72° C. for 30 sec followed by 28 cycles of 94° C. for 30 sec, 65° C. (MGMT region I) or 67° C. (MGMT region II) for 30 sec, 72° C. for 30 sec. A final incubation at 72° C. for 5 min was followed by a holding step at 4° C.

Abscription master mix was composed of 148 mM HEPES pH 7.4, 148 mM KCl, 37 mM MgCl₂, 6 mM UpU, 6 mM GTP and 0.06 volume Abscriptase-II (RiboMed). An Abscription master mix volume of 2 μl was combined with 10 μl of PCR reaction. Beads were removed from the PCR tubes before addition of the Abscription master mix. Abscription was done for 30 min at 76.4° C. Abscription reactions were stopped by reducing the incubation temperature to 25° C. Samples were prepared by mixing 5 μl of Abscription reaction with 20 μl of water in a 384 well plate. Ten microliters of diluted samples were analyzed by LC-MS.

LC-MS readout was performed on a Waters LCT-Premier ESI-TOF spectrometer and 2795 HPLC with a 3 μm, 2.1 mm×20 mm Atlantis C18 column. Solvent A was HPLC grade water, 0.01% formic acid. Solvent B was HPLC grade acetonitrile, 0.01% formic acid. The separation was performed over 8 min with a linear gradient of 0-21.6% Solvent B. An m/z range of 200 to 1000 was scanned. Abscript peaks were extracted from the total ion current data using singly charged and doubly charged m/z values of 446.8 (doubly charged UUG) and 894.2 (singly charged UUG). Signal intensities were the sums of the signals from singly charged and doubly charged ions represented as the areas under the chromatographic peaks.

### IDH1 R132H Determination

AluI digested FFPE DNA was subjected to CAP Format II using IDH1 R132H and wild type primers as described above. The target-specific forward primer for the R132H allele (5′ GGTAAAACCTATCATCATAGGTCA) was designed so that the R132H nucleotide (A) at 3′ end of the primer is complementary to the DNA template corresponding to the R132H SNP. The R132H-specific primer forms a 3′-terminal A:C mismatch with the wild type R132 complement. The A:C mismatch is sufficient to block amplification of the wild type target. The forward target-specific primer for the wild type allele R132 (5′ GGTAAAACCTATCATCATTGGTCG) forms a 3′ terminal G:T mismatch with the R132H allele. The G to T substitution 6 nt from the 3′ terminus in the wild type primer increases discrimination against the R132H allele. The reverse target-specific primer (5′ TGCAAAATCACATTATTGCCAAC) is used with both forward primers.

The PCR cycling conditions for the IDH1 allele amplifications were the same as for the methylation assays except that the Phase 1 annealing temperatures for the R132H primers and wt R132 primers were 60.7° C. and 58.5° C., respectively.

### Primer Design and Validation

CAP primer design involves first identifying target specific primer sequences and then screening in silico for primer dimer potential after appending the 5′ segment sequences. The target specific primer sequences were obtained using Primer Blast from the U. S National Center for Biotechnology Information web site (http://ncbi.nlm.nih.gov/tools/primer-blast). The PCR product size options was set to 40 Min and 200 Max to match the amplifiable DNA size range expected when working with FFPE or cell free DNA.

A total of 4 primers were used in the fluorescence detection assay. The APC producing the 11-mer is larger than the APC for the 3-mer and is most efficiently constructed in a two phase PCR with four primers. In Phase I, the forward primer had a truncated APC segment (APC-DN) appended to its 5′ end. The reverse primer had a Universal reverse segment linked to its 5′ end. These primers were used to create a tagged amplicon in Phase I. A third and fourth primer called the APC completion primer and the Universal reverse primer were responsible for amplification in Phase 2.

A text editor was used to paste the APC-DN segment sequence to the 5′ end of one of the target primers. The sequence of the Universal reverse segment was pasted to the 5′ end of the reverse primer. Each composite primer sequence was tested in conventional primer development software (e.g. Oligo Analyzer® program, IDT, IA, USA, http://www.idtdna.com/Scitools) for the potential to form self-dimers. Potential dimers with an annealed 3′ end >3 nt are rejected. Forward and reverse composite primers that passed the self-dimer test were further tested for formation of heterodimers. Potential heterodimers with an annealed 3′ end longer than 3 nt were rejected.

Candidate primer pairs were subjected to a Phase 1 annealing temperature gradient PCR followed by Phase 2 amplification with the APC-completion primer and the Universal reverse primer. DNA samples of 150 genomic copies per PCR were amplified in parallel with no-DNA controls for 30 PCR cycles. The completed reactions were subjected to Abscription for 15 cycles. Primers that produced sufficiently intense fluorescence signal and low background were accepted and assigned an optimal Phase 1 annealing temperature.

Only two primers are needed for the LC-MS based detection. The APC-primer encoding a trinucleotide Abscript is short enough to order as a complete APC-primer. Sample amplifications were performed with a promoter-target-specific primer and a target-specific reverse primer with no additional tags. Text versions of the APC-primer and reverse primer were screened for primer dimer potential and then were tested for background level and signal intensity in an annealing temperature gradient experiment.

### Results

### Workflow

FIG. 1 outlines the bisulfate-free, MethylMeter DNA methylation assay. The method relies on the separation of methylated DNA from unmethylated fragments with the use of magnetic beads bearing a GST-MBD2 fusion protein. According to the overall scheme of the assay, DNA samples are first fragmented, usually by AluI restriction, to isolate a targeted segment of the CpG island from flanking regions that might bias the fractionation of unmethylated targets. The extent of AluI cleavage is verified by amplifying AluI digested samples and uncut controls using a primer pair that is sensitive to AluI cleavage due to an intervening AluI site. Due to the high frequency of AluI sites flanking the target, incomplete digests of the MGMT CpG island are adequate to unlink neighboring CpG islands based on the recovery of unmethylated targets from samples with AluI cleavage frequencies as low as 67%. For analysis of the IDHR132H mutation, an aliquot is removed prior to fractionation (FIG. 1A, Step 1b).

Fragmented target DNAs are separated into methylated and unmethylated fractions using MethylMagnet (Hashimoto et al., Epigenetics. 2:86-91 (2007)), a magnetic bead bound GST-MBD2-Me-CpG binding domain protein (FIG. 1A, step 1a). The affinity fractionation provides the specificity of the assay. Methylated target DNA binds to the magnetic beads, while unmethylated targets remain in the supernatant fraction. The amount of DNA in each fraction is measured and used to calculate the percentage methylation for that target.

The amount of target DNA in each fraction is determined using a combination of end point PCR and Abscription, or Coupled Abscription-PCR (CAP). The targeted CpG island segment is amplified using primers to attach an abortive promoter cassette (APC) to each amplicon. The promoter is activated in the course of copying the target (FIG. 1B). Signal molecules in the form of short, abortive RNA transcripts are generated after the PCR step by adding RNA precursors and an APC-specific RNA polymerase referred to as Abscriptase (FIG. 1B).

Two formats for the CAP assay have been developed, each with strengths and weakness. In Format I, 11-mer Abscripts are generated and quantified by measuring fluorescence from a molecular beacon (FIG. 1B, Steps 2b and 2c). The 11-mer Abscripts anneal to a single-stranded segment of loop in the molecular beacon (FIG. 2A). Extension of the annealed Abscript by a DNA polymerase (CAP DNA polymerase) opens the beacon and separates a fluorescein at the beacon 5′-end from a BHQ-1® quencher at the beacon 3′-end. See U.S. Pat. No. 8,211,644. The rate of fluorescence increase during the Abscription reaction is directly proportional to the number of input DNA molecules sampled in the PCR reaction (FIGS. 2B and 2C). In Format II, trinucleotide Abscripts are generated and then quantified by LC-Mass Spec (FIG. 1B, Steps 2a and 3a).

### Sensitivity of MethylMagnet Fractionation

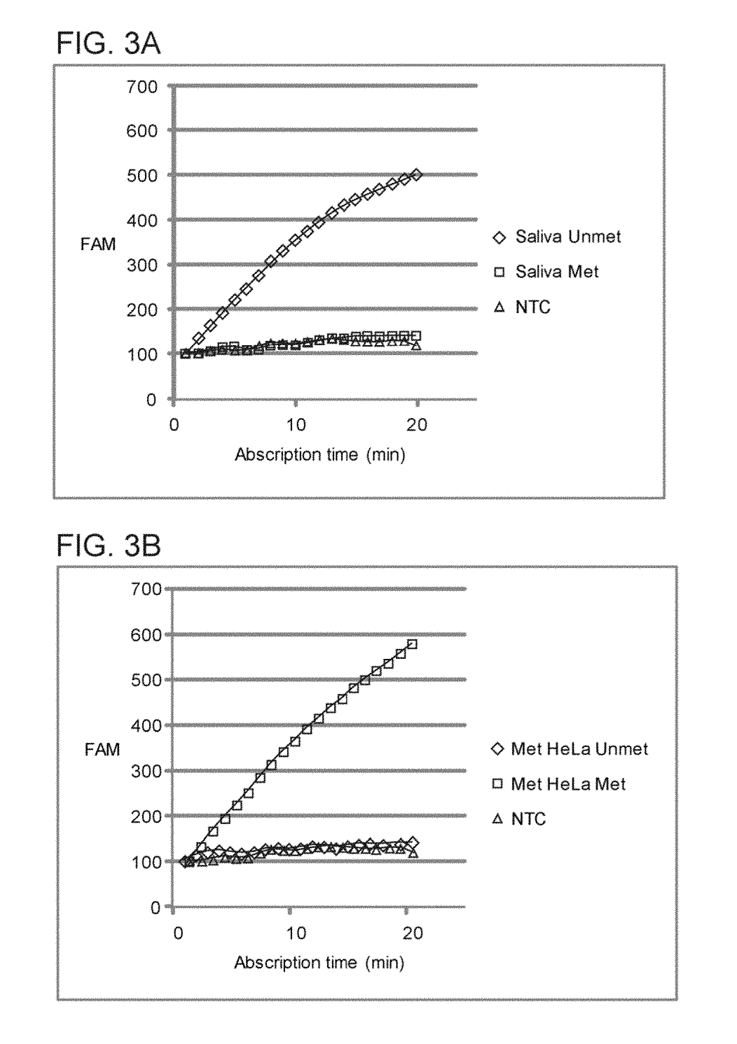

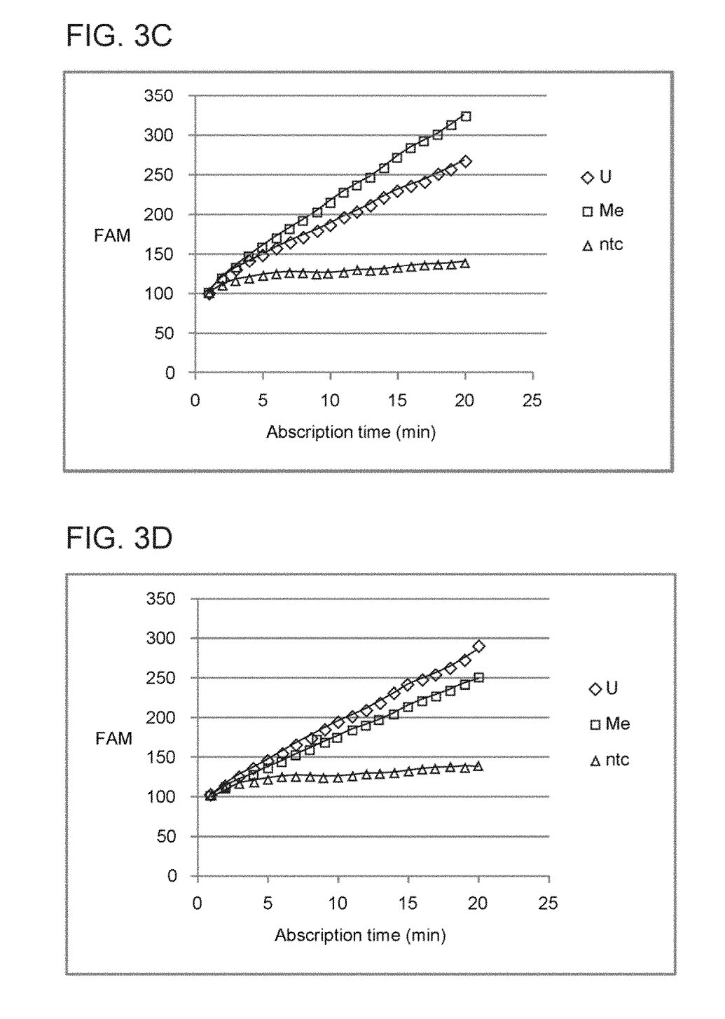

The sensitivity and accuracy of MethylMeter MBD-magnetic beads was determined at DNA samples between 6 and 30 genomic copies using small scale bead inputs (0.1 μl) and binding reaction volumes (2 μl). Fractionation of 6 copies of unmethylated HFE gene from normal (unmethylated) saliva DNA showed no evidence of nonspecific binding while 6 copies of the HFE gene from artificially methylated HeLa DNA showed complete HFE methylation (FIGS. 3A and 3B). The fractionation of 5 HeLa replicates was highly consistent with an average DNA methylation level of 98.7%±2.7.

The sampling error expected when distributing 6 genomic copies to the MethylMagnet binding reaction was reflected in the variability of the total amounts of DNA among the samples but not in the methylation results, where the fractions are homogeneous with respect to methylation, as summarized below in Table 1, which gives the quantitative results for the methylation analysis of HFE for normal DNA from saliva sediment verses artificially methylated HeLa and single measurements of SNRPN methylation at 6, 15 and 30 genomic copy inputs.

TABLE 1

| Sample | Percent HFE Methylation |
|---|---|
| Normal Saliva DNA 6 copies | 0 (n = 1) |
| Methylated HeLa DNA 6 copies | 97.7 ± 2.7 (n = 5) |
| Sample | Measured Total DNA Input |
| Methylated HeLa DNA 6 copies | 9.90 ± 3.5 (n = 5) |
| Sample | Percent SNRPN Methylation |
| HeLa DNA 6 copies | 59.9 (n = 1) |
| HeLa DNA 15 copies | 45.3 (n = 1) |
| HeLa DNA 30 copies | 60.2 (n = 1) |

The fractionation of the imprinted SNRPN gene at 6, 15 or 30 genomic copies of HeLa DNA was reasonably close to the 50% methylation level expected for an imprinted gene given the potential for sampling error to skew MethylMagnet inputs that are mixtures of methylated and unmethylated fragments.

### CAP

### Coupled Abscription PCR

In CAP, the copy number of each target in the bead (methylated) and supernatant fractions (unmethylated) are measured using a combination of end-point PCR, followed by real time fluorescence detection of amplicons by Abscription. The PCR phase generates amplicons that are each tagged with a specific APC. The APCs used here reiteratively generated either a 3-mer or an 11-mer Abscript.

A full length APC for the production of a trinucleotide Abscript is linked to a target-specific primer and used with a conventional reverse target-specific primer to tag amplicons. The single-stranded version of the APC is inactive for Abscription but becomes activated when a target is copied. The first three PCR cycles use an extended denaturation time (2 min) to compensate for the slow kinetics of denaturation of fully methylated DNAs. Aird et al., Genome Biol. 12:R18 (2011).

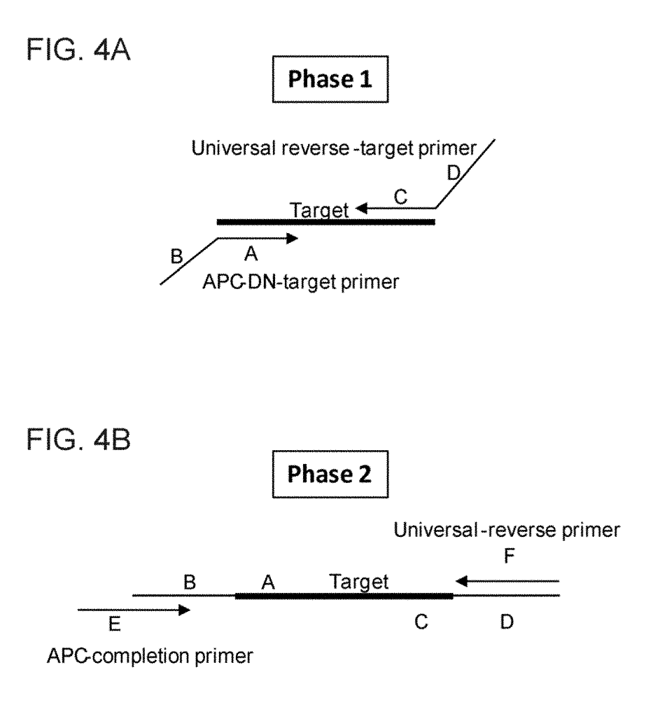

The APC for production of an 11-mer is too long to merge full-length with a target-specific primer. In this case a truncated downstream APC segment is linked to a target-specific primer (FIGS. 4A and 4B, sequences A and B). The incomplete APCs on the initial amplicons are made full length with an APC-completion primer as listed in Table 2.

The PCR program for the 11-mer encoding APC is broken into two phases that correspond to the different steps in APC assembly. Phase I consists of the first three cycles which are used to establish an amplicon with the truncated APC. This phase incorporates a 2.25 min denaturation time to accommodate the slow denaturation of fully methylated target. Phase I conditions are optimal for the APC-target-specific (forward) primer and a reverse target-specific primer with a 5′ universal sequence (FIGS. 4A and 4B sequences C and D). In Phase 2 an unmethylated copy of the target sequence exists and subsequent PCR cycles use a 5 sec denaturation time. Phase 2 conditions favor a second set of primers present at 0.5 μM (FIG. 4B sequences E and F). The APC-completion primer targets the truncated APC sequence on the amplicon and produces a complete active promoter (FIG. 4B, Phase 2, sequence E). The universal reverse primer is designed to anneal under the same conditions as the APC-completion primer and uncouples phase 2 amplification conditions from those of the reverse target-specific primer (FIG. 4B, Phase 2, sequence F). Phase 2 conditions are designed to maximize the production of amplicons with full length promoters at the expense of amplicons with the incomplete downstream promoter.

Table 2 below shows sequences of the target primers and their linkages to the APC-DN segment and the universal reverse segment.

TABLE 2
Primer Sequences

| | | | Target-specific Primers | |
|---|---|---|---|---|
| Gene Symbol* | Description | Gene ID | Forward | Reverse |
| MGMT | O-6- | 4255 | 5′-APC-DN-GCGCACC | 5′-UR-AGCGAGGCG |
| region I | methylguanine- | | GTTTGCGACTTGG-3′ | ACCCAGACACT-3′ |
| (SEQ ID NO: 39) | DNA | | (SEQ ID NO: 1) | (SEQ ID NO: 2) |
| | methyltransferase | | | |
| MGMT | O-6- | 4255 | 5′-APC-DN-CGGCTTGT | 5′-UR-CTGTGCGCC |
| region II | methylguanine- | | ACCGGCCGAAGG-3′ | TGACCCGGATG-3′ |
| (SEQ ID NO: 40) | DNA | | (SEQ ID NO: 3) | (SEQ ID NO: 4) |
| | methyltransferase | | | |
| HFE | hemochromatosis | 3077 | 5′-APC-DN-CGGCGCT | 5′-UR-CAGCCCTCG |
| (SEQ ID NO: 41) | | | TCTCCTCCTGATGC-3′ | GACTCACGCAG-3′ |
| | | | (SEQ ID NO: 5) | (SEQ ID NO: 6) |
| MAL | mal, T-cell | 4118 | 5′-APC-DN-GGATCCC | 5′-UR-CTCAGTGGA |
| (SEQ ID NO: 42) | differentiation | | AGCGCCGAACCAG-3′ | CGCGGAAGGGG-3′ |
| | protein | | (SEQ ID NO: 7) | (SEQ ID NO: 8) |
| SOWAHA | sosondowah | 134548 | 5′-APC-DN-GCCCGCA | 5′-UR-CCTTCACCA |
| (SEQ ID NO: 43) | ankyrin repeat | | GGGACCGCTTCAA-3′ | CCGCCACGTTGT-3′ |
| | domain family | | (SEQ ID NO: 9) | (SEQ ID NO: 10) |
| | member A | | | |
| IDH1 R132 wt | isocitrate | 3417 | 5′-APC-DN-GGTAAAA | 5′-UR-TGCAAAATC |
| (SEQ ID NO: 44) | dehydrogenase | | CCTATCATCATTGGT | ACATTATTGCCAA |
| | (NADP(+)) 1, | | CG-3′ | C-3′ |
| | cytosolic | | (SEQ ID NO: 11) | (SEQ ID NO: 12) |
| IDH1 R1 32H | isocitrate | 3417 | 5′-APC-DN-GGTAAAA | 5′-UR-TGCAAAATC |
| SNP | dehydrogenase | | CCTATCATCATAGGT | ACATTATTGCCAA |
| (SEQ ID NO: 45) | (NADP(+)) 1, | | CA-3′ | C-3′ |
| | cytosolic | | (SEQ ID NO: 13) | (SEQ ID NO: 14) |
| | AluI cleavage | | 5′-APC-DN-AGGCACT | 5′-UR-GAGGGCTGC |
| | validation | | CCCTCACGGGGTC-3′ | GGGCGAACTAG-3′ |
| | | | (SEQ ID NO: 15) | (SEQ ID NO: 16) |

Universal sequences

| | |
|---|---|
| APC-DN segment (B) | 5′-CTTACAATGCATGCTATAATACCACTATCGGTGCTT |
| | TAAAATTCNN-3′ |
| | (SEQ ID NO: 17) |
| APC-completion primer (E) | 5′-CCTTTAAAGAAAATTATTTTAAATTTATGTTTGACA |
| | GATCTTACAATGCATGCTATAATACCA-3′ |
| | (SEQ ID NO: 18) |
| Universal reverse segment | 5′-AGTGAATAAGGCTTGCCCTGACGN-3′ |
| (UR) (D) | (SEQ ID NO: 19) |
| Universal reverse primer (F) | 5′-AGTGAATAAGGCTTGCCCTGACGA-3′ |
| | (SEQ ID NO: 20) |

Primers for trinucleotide Abscripts (LC-MS detection)

| | | |
|---|---|---|
| MGMT region I | 5′-GCAAAAAAAAAAAAAAAAAAA | 5′-AGCGAGGCGACCCAGACAC |
| (SEQ ID NO: 39) | AAAAATGTATAATGGGAACTTGC | T -3′ |
| | GCACCGTTTGCGACTTGG-3′ | (SEQ ID NO: 22) |
| | (SEQ ID NO: 21) | |
| MGMT region II | 5′-GCAAAAAAAAAAAAAAAAAAA | 5′-TGTGCGCCTGACCCGGATGC- |
| (SEQ ID NO: 40) | AAAAATGTACAATGGGAACTTTG | 3′ |
| | CGGCTTGTACCGGCCGAAGG-3′ | (SEQ ID NO: 24) |
| | (SEQ ID NO: 23) | |

*Gene Symbol refers to the short-form abbreviation approved by the HUGO Gene Nomenclature Committee. See world wide web at genenames (dot) org

As the PCR and Abscription steps are performed sequentially, it is important to choose a PCR endpoint that does not produce enough amplicons to saturate the downstream Abscription reaction. Table 3 gives guidelines for the appropriate number of PCR cycles for MGMT, HFE, MAL and SOWAHA detection at different DNA inputs into the MethylMagnet binding reaction. In the case of damaged DNA from FFPE samples, the DNA input is based on amplifiable DNA. The amount of amplifiable DNA in a sample is known before the DNA fractionation step because the AluI digests are calibrated in terms of amplifiable genomic copies/μl during the evaluation of AluI cleavage. It is especially important to determine the amplifiable DNA content when processing FFPE DNA because formalin fixed DNA is heavily damaged and the total DNA concentration is usually significantly higher than the amplifiable fraction.

TABLE 3
Endpoint PCR cycles verses DNA input

| Endpoint cycle # | MethylMagnet DNA input (20 μl) | |
|---|---|---|
| 31 | ≦10 | ng |
| 29 | 10 to 40 | ng |
| 27 | 40 to 200 | ng |
| 25 | 200 to 1000 | ng |

### MGMT Promoter Methylation

CAP based detection is more sensitive than other PCR based methods in part because each amplicon produces multiple signals. CAP with LC-MS detection of trinucleotides is 100-1000 fold more sensitive than comparable qPCR on undamaged DNA samples. McCarthy et al. (2012), supra. The enhanced sensitivity makes CAP detection highly effective with heavily damaged formalin fixed DNA samples. MethylMeter was tested with fluorescent detection on a set of split FFPE primary glioma samples that had been fixed with formalin or RCL2. Pulverer et al., Clin Neuropathol. 33:50-60 (2014). FFPE samples are prone to analytical failure because the fixation process introduces DNA damage in the form of crosslinks and strand breaks. RCL2 fixation is expected to have a higher success rate because much less DNA damage is induced upon fixation. Delfour et al., J Mol Diagn. 8:157-69 (2006); Preusser et al., Brain Pathol. 20:1010-20 (2010). The previous MGMT promoter methylation analysis of the FFPE set by methylation sensitive restriction enzymes (MSRE) failed at AIT but the RCL2 samples were successfully analyzed. Pulverer et al., Clin Neuropathol. 33:50-60 (2014). To test the sensitivity of the MethylMeter molecular beacon assay, AIT FFPE samples were analyzed and the accuracy evaluated with the available MSRE data from the RCL2 samples.

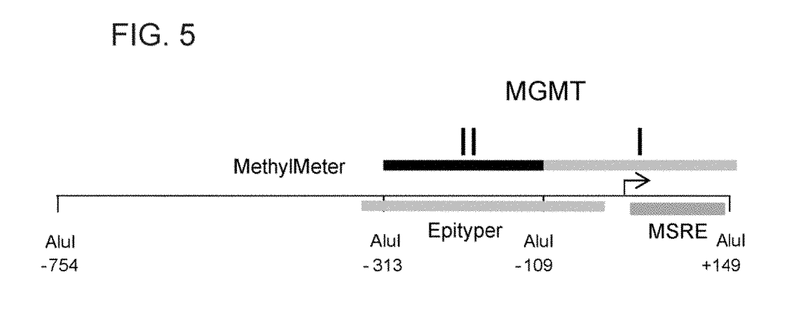

The MGMT CpG island is broken into two targets; Region I is defined by AluI cleavage sites situated at −109 and +149 from the transcription start site (TSS) and Region II is defined by sites at −313 and −109. FFPE DNAs from primary gliomas were purified and processed as described in Materials & Methods. All results were calibrated and expressed as genomic copies/μl using titrations of HeLa DNA processed in CAP reactions run in parallel with the MethylMagnet fractions. FIG. 5 shows comparisons of the molecular beacon based fluorescence detection to an alternative CAP method that uses HPLC-mass spectroscopy (LC-MS) of trinucleotide Abscripts. McCarthy et al., (2012), supra. Methylation measurements were targeted to the downstream segment of the MGMT CpG island that includes a segment that had previously been analyzed by MSRE on RCL2 samples (Region I at coordinates +109 to +149 relative to the TSS, FIG. 5). Pulverer et al. (2014), supra. The methylation cutoff for the MSRE detection was set at 2 standard deviations above the average methylation level of normal brain samples. Pulverer et al. (2014), supra. A cutoff of 10% for the MethylMeter LC-MS and molecular beacon detection maximized the agreement among these methods and MSRE.

Overall there was good agreement between the CAP LC-MS and molecular beacon methods considering they were performed 3 years apart with the same DNA samples (Table 4). The single discordant result between the molecular beacon and the LC-MS detection for sample 15 was resolved in favor of the molecular beacon detection based on survival data. A comparison of the MethylMeter beacon results against the MSRE results showed four discordant results (Table 4). The apparent disagreement over sample 8 might not be real given that it is close to the cutoffs for both methods. Survival data for samples 19, 21 and 31 were more consistent with the MethylMeter data.

The same set of RCL2 fixed samples were analyzed by AIT using the bisulfate and mass spec based Epityper® method. Ehrich et al., Proc Natl Acad Sci USA. 102:15785-90 (2005). The region analyzed by Epityper® overlapped with Region I and Region II but did not overlap with the region analyzed by MSRE (FIG. 5). There were only two discordant results between the Epityper® and MethylMeter assignments. The survival data for sample 31 was more consistent with the MethylMeter assignment. There was no survival data available for sample 8 to resolve the disagreement.

A second MGMT validation was performed focusing on a set of 26 recurrent glioblastoma tumors from frozen tumor tissue for which mRNA data were also obtained. Cloughesy et al., Submitted for publication (2015). These samples were previously analyzed for MGMT methylation by undisclosed methods at multiple clinical study centers. The mRNA abundance data were used to resolve discordant results with the clinical site methylation assays. DNA methylation levels were measured for MGMT CpG island Regions I and II. Methylation of either of these regions can reduce MGMT expression. Bady et al., Acta Neuropathol. 124:547-60 (2012); Everhard et al., Neuro Oncol. 11:348-56 (2009); Shah et al., PLoS One 6:e16146 (2011). The methylation assignments were always consistent between the two regions (Table 5). Comparison of the MethylMeter results to the methylation assignments from the clinical sites showed four disagreements. In the three discordant cases where MGMT mRNA data were available, the RNA levels were consistent with the MethylMeter methylation assignments (Table 5, samples 40, 44, 58).

There were two cases (samples 39 and 57) where an unmethylated call by MethylMeter was matched with a low RNA level. This kind of result is not unexpected because an unmethylated status does not guarantee expression. There were 2 cases (samples 40 and 58) where the clinical sites assigned a methylated status to a sample that showed MGMT expression. This kind of result implies that their methylation assignment is incorrect.

### GliomaSTRAT and IDH1 SNP Detection

The high sensitivity of the CAP based detection and the nondestructive nature of MethylMeter maximizes the number of genes that can be analyzed from a given sample. In addition to methylation analysis, either MethylMagnet fraction potentially can be used for other types of analysis. This was demonstrated by analyzing glioma samples for the CpG Island Methylator Phenotype and the IDH1 R132H SNP that is often used as an alternative marker to infer this phenotype in the absence of methylation data. Noushmehr et al., Cancer Cell 17:510-22 (2010); Turca et al., Nature 483:479-83 (2012).

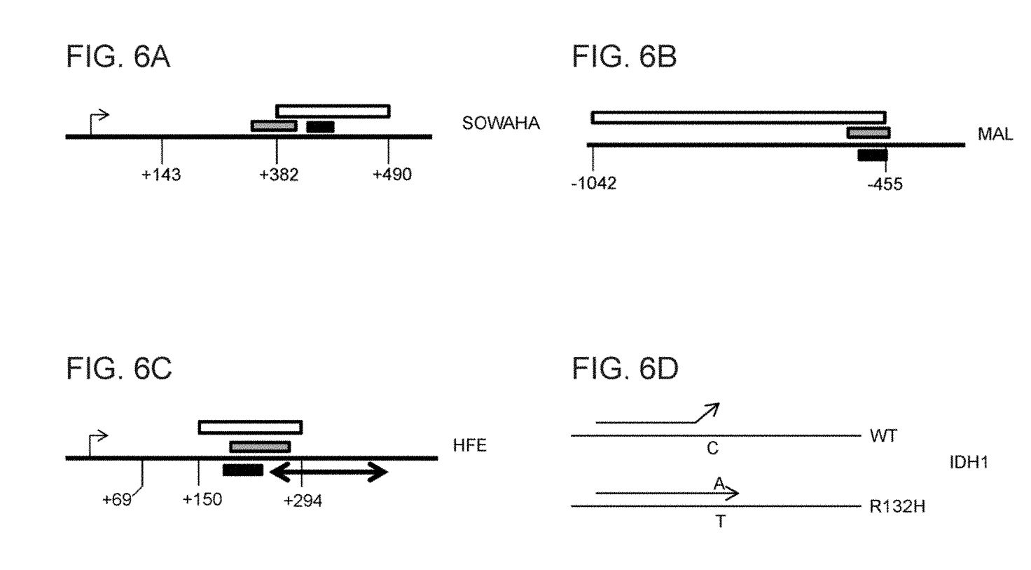

DNA methylation analysis of gliomas demonstrated that a glioma CpG island methylator phenotype (G-CIMP) exists that can identify patients with improved prognosis within histological grades 2, 3 and 4. Noushmehr et al. (2010), supra. In the majority of cases, the methylated G-CIMP was caused by gain of function mutations in the isocitrate dehydrogenase gene (IDH1) which result in remodeling of the epigenome and production of G-CIMP associated changes in transcription. Turca et al. (2012), supra. The original 8-gene G-CIMP methylation signature to 3 genes (HFE, MAL and SOWAHA) using MethylMeter with LC-MS detection (data not shown). The CIMP targets were defined by AluI cleavage sites located at the following coordinates relative to the TSS: −46 to +146 (HFE); +154 to +298 (MAL) and −1046 to −455 (SOWAHA). FIG. 6A-D and Tables 6 and 7 shows the results of the 3 gene test applied to tumor samples diagnosed as WHO grade 2 (low grade, Table 6) and high grade 4 glioblastoma (Table 7). A methylated call for at least 2 of the 3 CIMP CpG islands caused a G-CIMP-plus assignment. The G-CIMP results were concordant with the IDH1 R132H results as expected and the combined methylation and SNP results agreed with the clinical diagnoses.

TABLE 6
Grade 2

| Biomarker | Result | Tumor Phenotype |
|---|---|---|
| Me-MGMT | 23% | TMZ responder |
| Me-HFE | 27% | CIMP + (LGG) |
| Me-MAL | 55% | |
| Me-SOWAHA | 28% | |
| IDH1-R132H | Positive | LGG |

TABLE 7
GMB

| Biomarker | Result | Tumor Phenotype |
|---|---|---|
| Me-MGMT | 0% | TMZ Non-responder |
| Me-HFE | 0% | CIMP − (LGG) |
| Me-MAL | 0% | |
| Me-SOWAHA | 6% | |
| IDH1-R132H | Negative | HGG |

### Example 3

### Methylation Detection in FFPE Samples

The following additional primer pairs summarized in Table 8, below, have been designed and validated for detection of methylation using the methods described herein.

TABLE 8

| | | | Target-specific Primers | |
|---|---|---|---|---|
| Gene Symbol* | Description | Gene ID | Forward | Reverse |
| CDH1 | cadherin 1 | 999 | 5′-APC-DN-GTCAGTT | 5′-UR-GAATGCGTCCC |
| (SEQ ID NO: 46) | | | CAGACTCCAGCC-3′ | TCGCAAGT-3′ |
| | | | (SEQ ID NO: 25) | (SEQ ID NO: 26) |
| DAPK1 | death associated | 1612 | 5′-APC-DN-TCGGAGT | 5′-UR-GGAGGGAACAA |
| (SEQ ID NO: 47) | protein kinase 1 | | GTGAGGAGGAC-3′ | AGTCCC-3′ |
| | | | (SEQ ID NO: 27) | (SEQ ID NO: 28) |
| HOXA2 | homeobox A2 | 3199 | 5′-APC-DN-TTGTCCT | 5′-UR-CAGAACCCGGA |
| (SEQ ID NO: 48) | | | TGTCGCTCTGGT-3′ | AGCAAACA-3′ |
| | | | (SEQ ID NO: 29) | (SEQ ID NO: 30) |
| KCTD5 | potassium | 54442 | 5′-APC-DN-TCTGAGT | 5′-UR-AGCTGTTCTGA |
| (SEQ ID NO: 49) | channel | | GATCGTGGTGCAG-3′ | GCCAAGCC-3′ |
| | tetramerization | | (SEQ ID NO: 31) | (SEQ ID NO: 32) |
| | domain | | | |
| | containing 5 | | | |
| MLH1 | mutL homolog 1 | 4292 | 5′-APC-DN-CTGGTTG | 5′-UR-TACCAGTGCAT |
| (SEQ ID NO: 50) | | | CGTAGATTCCTGTC- | GGAGGTGTTGCT-3′ |
| | | | 3′ | (SEQ ID NO: 34) |
| | | | (SEQ ID NO: 33) | |
| RB | RB | 5925 | 5′-APC-DN-ACTGTGA | 5′-UR-TTATCCTTGGGG |
| (SEQ ID NO: 51) | transcriptional | | AACTGCAGCCAG-3′ | CGTTTGGG-3′ |
| | corepressor 1 | | (SEQ ID NO: 35) | (SEQ ID NO: 36) |
| VTRNA2-1 | vault RNA 2-1 | 100126299 | 5′-APC-DN-TCCTGGA | 5′-UR-TGGAGAGAACC |
| (SEQ ID NO: 52) | | | GGGACTCTCAGT-3′ | CCGAAAAGC-3′ |
| | | | (SEQ ID NO: 37) | (SEQ ID NO: 38) |

*Gene Symbol refers to the short-form abbreviation approved by the HUGO Gene Nomenclature Committee. See world wide web at genenames (dot) org

What is claimed is:

1. A method for detecting CpG island methylation comprising:
a) separating methylated DNA comprising at least one CpG island from unmethylated DNA in a sample; and
b) performing Coupled Abscription PCR to detect the presence of the at least one CpG island nucleotide sequence in the methylated DNA,
thereby detecting CpG island methylation.

2. The method of claim 1, wherein the Coupled Abscription PCR uses three primers.

3. The method of claim 2, therein the three primers comprise:
a) a forward target-specific primer that has a truncated Abortive Promoter Cassette (APC) sequence at its 5′ end;
b) a reverse target-specific primer for creating an amplicon that contains a duplex inactive APC; and
c) an APC primer that overlaps with the 5′ end of the truncated APC sequence.

4. The method of claim 1, wherein the Coupled Abscription PCR uses four primers.
a) a forward target-specific primer that has a truncated APC sequence at its 5′ end;
b) a reverse target-specific primer comprising a universal primer sequence at the 5′ end of a target-specific priming sequence;
c) an APC completion primer that overlaps with the 5′ end of the truncated APC sequence;
d) a universal reverse primer.

5. The method of claim 1, wherein the presence of the at least one CpG island nucleotide sequence is detected by fluorescence.

6. The method of claim 5, wherein fluorescence is produced by opening of a molecular beacon.

7. The method of claim 6, wherein the molecular beacon is opened by an Abscript produced during Coupled Abscription PCR.

8. The method of claim 1, wherein the presence of the at least one CpG island nucleotide sequence is detected by mass spectrometry.

9. The method of claim 1, wherein the CpG island has a sequence selected from the group consisting of: SEQ ID NOs:39-52.

10. The method of claim 1, wherein at least one CpG island nucleotide sequence comprises at least two CpG island nucleotide sequences.

11. The method of claim 10, wherein the at least two CpG island nucleotide sequences are selected from the consisting of: SEQ ID NOs:41-43.

12. The method of claim 1, further comprising detecting a Single Nucleotide Polymorphism in the sample.

13. A method for to assembling and fusing a full length Abortive Promoter Cassette (APC) to a target nucleic acid during PCR amplification of the target comprising the steps of:
a) providing a forward target-specific primer that has a truncated APC sequence at its 5′ end;
b) providing a reverse target-specific primer for creating an amplicon that contains a duplex inactive APC;
c) providing an APC primer that overlaps with the 5′ end of the truncated APC sequence; and
d) amplifying the target with the three primers,
thereby assembling and fusing a full-length APC to a target nucleic acid during PCR amplification of the target.

14. The method of claim 6, wherein the primer of step a) is present at a lower concentration than primers of steps b) and c) during the amplifying step.

15. The method of claim 13, wherein the target comprises a CpG island.

16. The method of claim 15, wherein the CpG island has a sequence selected from the group consisting of: SEQ ID NOs:39-52.

17. A method for to assembling and fusing a full length Abortive Promoter Cassette (APC) to a target nucleic acid during PCR amplification of the target comprising the steps of:
a) providing a forward target-specific primer that has a truncated APC sequence at its 5′ end;
b) providing a reverse target-specific primer comprising a universal primer sequence at 5′ end of a target-specific priming sequence;
c) providing an APC completion primer that overlaps with the 5′ end of the truncated APC sequence;
d) providing a universal reverse primer; and
e) amplifying the target with the four primers,
thereby assembling and fusing a full-length APC to a target nucleic acid during PCR amplification of the target.

18. The method of claim 17, wherein the target comprises a CpG island.

19. The method of claim 18, wherein the CpG island has a sequence selected from the group consisting of: SEQ ID NOs:39-52.

20. The method of claim 17, wherein primers of steps c) and d) are present at a lower concentration than primers of steps a) and b) during the amplifying step.


SEQUENCE LISTING (52 sequences)

SEQ ID NO: 1 - DNA, 66 bp, Artificial Sequence
  synthetic primer
cttacaatgcatgctataataccactatcggtgctttaaaattccggcgcaccgtttgcgacttgg

SEQ ID NO: 2 - DNA, 44 bp, Artificial Sequence
  synthetic primer
agtgaataaggcttgccctgacgaagcgaggcgacccagacact

SEQ ID NO: 3 - DNA, 66 bp, Artificial Sequence
  synthetic primer
cttacaatgcatgctataataccactatcggtgctttaaaattccgcggcttgtaccggccgaagg

SEQ ID NO: 4 - DNA, 44 bp, Artificial Sequence
  synthetic primer
agtgaataaggcttgccctgacgactgtgcgcctgacccggatg

SEQ ID NO: 5 - DNA, 67 bp, Artificial Sequence
  synthetic primer
cttacaatgcatgctataataccactatcggtgctttaaaattccgcggcgcttctcctcctgatgc

SEQ ID NO: 6 - DNA, 44 bp, Artificial Sequence
  synthetic primer
agtgaataaggcttgccctgacgacagccctcggactcacgcag

SEQ ID NO: 7 - DNA, 66 bp, Artificial Sequence
  synthetic primer
cttacaatgcatgctataataccactatcggtgctttaaaattccgggatcccagcgccgaaccag

SEQ ID NO: 8 - DNA, 44 bp, Artificial Sequence
  synthetic primer
agtgaataaggcttgccctgacgactcagtggacgcggaagggg

SEQ ID NO: 9 - DNA, 66 bp, Artificial Sequence
  synthetic primer
cttacaatgcatgctataataccactatcggtgctttaaaattccggcccgcagggaccgcttcaa

SEQ ID NO: 10 - DNA, 45 bp, Artificial Sequence
  synthetic primer
agtgaataaggcttgccctgacgaccttcaccaccgccacgttgt

SEQ ID NO: 11 - DNA, 70 bp, Artificial Sequence
  synthetic primer
cttacaatgcatgctataataccactatcggtgctttaaaattccgggtaaaacctatcatcattggtcg

SEQ ID NO: 12 - DNA, 47 bp, Artificial Sequence
  synthetic primer
agtgaataaggcttgccctgacgatgcaaaatcacattattgccaac

SEQ ID NO: 13 - DNA, 70 bp, Artificial Sequence
  synthetic primer
cttacaatgcatgctataataccactatcggtgctttaaaattccgggtaaaacctatcatcataggtca

SEQ ID NO: 14 - DNA, 47 bp, Artificial Sequence; identical to SEQ ID NO: 12
  synthetic primer

SEQ ID NO: 15 - DNA, 66 bp, Artificial Sequence
  synthetic primer
cttacaatgcatgctataataccactatcggtgctttaaaattccgaggcactccctcacggggtc

SEQ ID NO: 16 - DNA, 44 bp, Artificial Sequence
  synthetic primer
agtgaataaggcttgccctgacgagagggctgcgggcgaactag

SEQ ID NO: 17 - DNA, 46 bp, Artificial Sequence
  synthetic primer
cttacaatgcatgctataataccactatcggtgctttaaaattcnn

SEQ ID NO: 18 - DNA, 63 bp, Artificial Sequence
  synthetic primer
cctttaaagaaaattattttaaatttatgtttgacagatcttacaatgcatgctataatacca

SEQ ID NO: 19 - DNA, 24 bp, Artificial Sequence
  synthetic primer
agtgaataaggcttgccctgacgn

SEQ ID NO: 20 - DNA, 24 bp, Artificial Sequence
  synthetic primer
agtgaataaggcttgccctgacga

SEQ ID NO: 21 - DNA, 62 bp, Artificial Sequence
  synthetic primer
gcaaaaaaaaaaaaaaaaaaaaaaaatgtataatgggaacttgcgcaccgtttgcgacttgg

SEQ ID NO: 22 - DNA, 20 bp, Artificial Sequence
  synthetic primer
agcgaggcgacccagacact

SEQ ID NO: 23 - DNA, 64 bp, Artificial Sequence
  synthetic primer
gcaaaaaaaaaaaaaaaaaaaaaaaatgtacaatgggaactttgcggcttgtaccggccgaagg

SEQ ID NO: 24 - DNA, 20 bp, Artificial Sequence
  synthetic primer
tgtgcgcctgacccggatgc

SEQ ID NO: 25 - DNA, 65 bp, Artificial Sequence
  synthetic primer
cttacaatgcatgctataataccactatcggtgctttaaaattccagtcagttcagactccagcc

SEQ ID NO: 26 - DNA, 43 bp, Artificial Sequence
  synthetic primer
agtgaataaggcttgccctgacgagaatgcgtccctcgcaagt

SEQ ID NO: 27 - DNA, 64 bp, Artificial Sequence
  synthetic primer
cttacaatgcatgctataataccactatcggtgctttaaaattccttcggagtgtgaggaggac

SEQ ID NO: 28 - DNA, 41 bp, Artificial Sequence
  synthetic primer
agtgaataaggcttgccctgacgcggagggaacaaagtccc

SEQ ID NO: 29 - DNA, 65 bp, Artificial Sequence
  synthetic primer
cttacaatgcatgctataataccactatcggtgctttaaaattcccttgtccttgtcgctctggt

SEQ ID NO: 30 - DNA, 43 bp, Artificial Sequence
  synthetic primer
agtgaataaggcttgccctgacgacagaacccggaagcaaaca

SEQ ID NO: 31 - DNA, 66 bp, Artificial Sequence
  synthetic primer
cttacaatgcatgctataataccactatcggtgctttaaaattccgtctgagtgatcgtggtgcag

SEQ ID NO: 32 - DNA, 43 bp, Artificial Sequence
  synthetic primer
agtgaataaggcttgccctgacgaagctgttctgagccaagcc

SEQ ID NO: 33 - DNA, 67 bp, Artificial Sequence
  synthetic primer
cttacaatgcatgctataataccactatcggtgctttaaaattccgctggttgcgtagattcctgtc

SEQ ID NO: 34 - DNA, 47 bp, Artificial Sequence
  synthetic primer
agtgaataaggcttgccctgacgataccagtgcatggaggtgttgct

SEQ ID NO: 35 - DNA, 65 bp, Artificial Sequence
  synthetic primer
cttacaatgcatgctataataccactatcggtgctttaaaattcagactgtgaaactgcagccag

SEQ ID NO: 36 - DNA, 44 bp, Artificial Sequence
  synthetic primer
agtgaataaggcttgccctgacgattatccttggggcgtttggg

SEQ ID NO: 37 - DNA, 65 bp, Artificial Sequence
  synthetic primer
cttacaatgcatgctataataccactatcggtgctttaaaattccgtcctggagggactctcagt

SEQ ID NO: 38 - DNA, 44 bp, Artificial Sequence
  synthetic primer
agtgaataaggcttgccctgacgatggagagaaccccgaaaagc

SEQ ID NO: 39 - DNA, 259 bp, Homo sapiens
agctccgcccccgcgcgccccggccccgcccccgcgcgctctcttgcttttctcaggtcctcggctccgccccgctctagaccccgccccacgccgccatccccgtgcccctcggccccgcccccgcgccccggatatgctgggacagcccgcgcccctagaacgctttgcgtcccgacgcccgcaggtcctcgcggtgcgcaccgtttgcgacttggtgagtgtctgggtcgcctcgctcccggaagagtgcggagct

SEQ ID NO: 40 - DNA, 206 bp, Homo sapiens
agctgggaaggcgccgcccggcttgtaccggccgaagggccatccgggtcaggcgcacagggcagcggcgctgccggaggaccagggccggcgtgccggcgtccagcgaggatgcgcagactgcctcaggcccggcgccgccgcacagggcatgcgccgacccggtcgggcgggaacaccccgcccctcccgggctccgccccagc

SEQ ID NO: 41 - DNA, 1283 bp, Homo sapiens
attaaatcagtgtgtctggggttaggagcaggcctcaatatgtttaatcattctccagataatcccaatactgtaaagtttgtgaaacacttgtcagataattcaattatgaaggctgtggaaggtgtttcagtaggatctaattggttaatgttatgacttaattaatttgaatcaaaaaacaaaatgaaaaagctttatatttctaagtcaaataagacataagttggtctaaggttgagataaaatttttaaatgtatgattgaattttgaaaatcataaatatttaaatatctaaagttcagatcagaacattgcgaagctactttccccaatcaacaacaccccttcaggatttaaaaaccaagggggacactggatcacctagtgtttcacaagcaggtaccttctgctgtaggagagagagaactaaagttctgaaagacctgttgcttttcaccaggaagttttactgggcatctcctgagcctaggcaatagctgtagggtgacttctggagccatccccgtttccccgccccccaaaagaagcggagatttaacggggacgtgcggccagagctggggaaatgggcccgcgagccaggccggcgcttctcctcctgatgcttttgcagaccgcggtcctgcaggggcgcttgctgcgtgagtccgagggctgcgggcgaactaggggcgcggcgggggtggaaaaatcgaaactagctttttctttgcgcttgggagtttgctaactttggaggacctgctcaaccctatccgcaagcccctctccctactttctgcgtccagaccccgtgagggagtgcctaccactgaactgcagataggggtccctcgccccaggacctgccccctcccccggctgtcccggctctgcggagtgacttttggaaccgcccactcccttcccccaactagaatgcttttaaataaatctcgtagttcctcacttgagctgagctaagcctggggctccttgaacctggaactcgggtttatttccaatgtcagctgtgcagttttttccccagtcatctccaaacaggaagttcttccctgagtgcttgccgagaaggctgagcaaacccacagcaggatccgcacggggtttccacctcagaacgaatgcgttgggcggtgggggcgcgaaagagtggcgttggggatctgaattcttcaccattccacccacttttggtgagacctggggtggaggtctctagggtgggaggctcctgagagaggcctacctcgggcct

SEQ ID NO: 42 - DNA, 1250 bp, Homo sapiens
ttaaggcaaaaaataaaaaatagtgaaacagagaaacaaaacatgaaacaccggcagtcaacaggcaggcaaagaacctgggggtgggggtagcagcggtcccaccctcaaaaggcccgggctgcccagaccaagagaaagcgatgaatctcttctggtaacgtcccttcctgtcgcatggattcaaggccgacctgccccagcaccaccaccagcagccttctgctggggccggcacagctgggagcaacctcctactctcaggcagacgcgcagcaccaagcagagaggcccggtgcaggatcccagcgccgaaccagcgccggctcagtggacgcggaaggggccggcggccgcggccggtcccatcccccactgcagacccccagcctgtggcggtggtccagttccgccaggaaaccgccgcctggagctgtgggtcgcgcacattaacgcatccagcggaaaaatgaaggagacccaaattcaaagttaaagtaatggtgacccgagaggtgccttgatgagaaggtttggggtcccggttactgatggttatcattcttacgagatgctggtcacctacgaagggagaaaggcacgaggagcgcctgaccaaagtggttttgccctgcttcccgcaagaggtggcacccacggctggaacgcaggagtcagacccacagtccccagctctggacgcccgcagcggggcctcgaagaggttcagggcggtgcccgcggcgctcgggccgggtctcccggggcgtggggcggggggcggggttgggcggcggccggggctcctccctcttctgccccgggctcccctgctcttaacccgcgcgcgggggcgcccaggccactgggctccgcggagccagcgagaggtctgcgcggagtctgagcggcgctcgtcccgtcccaaggccgacgccagcacgccgtcatggcccccgcagcggcgacggggggcagcaccctgcccagtggcttctcggtcttcaccaccttgcccgacttgctcttcatctttgagtttgtgagtggctcctggccggggaagggacggggtgggctgagccgtgcgctctctcgggcgcccagcacagctgtcggacgggatccgctagctgcgcaggttctgggagcatcggggcagcaggcgcagggcggggactaagccagggaagtcccctcccacctccggtcctttgtgcccttctagaccaacagaatgaggggaacag

SEQ ID NO: 43 - DNA, 2759 bp, Homo sapiens
attctcttaccatgagaaaattgcttttgaaaacgtctctctaaacctagagtaacgtgcattgccgcgcagagtgaattggttttgctgtaaatctcatgaagccgttttcctgcatgaaacacagtaaaaaaaaaaaaaaactggagtggtattgacgacctttaaaaaaaaaagattgttctgaaatcatcgcttggtgttttgcagtggttaaatgtttattaaaggaataaaaatccattcctccttggcggaccgcggcgtccctagggaacacacccacctagcaaccgagaccagagctggagcgctgcgcctttaattctggggcgggcggggggcggcgggaatttctgctccggttccctctgcatcgcgggggcagctgtcacctgtaaggcgagcgcggcgcgggggatggaagcaggcgcccattggacagaatacaaaggcagaaacctaccccgaaggccggggcccggcggggcgctgggggtgggcgctccccgcggcccccggcgcccttcgccccgagacgccccggcccagcggcactggcgcgaccgaggtccagcttcggggacacgcccggctggccgcggggaaggcaccaggtgagcgcggccgcgcctcccggaaccccgctcccgcgcgtcccgcggcgacgcggcgcccacccgccccgggagccaggaacccagggccccaccatggcgctggccgccgccgccgccgctgcggctgccggggtgagccaggcggcggtgctgggcttcctgcaggagcacggcgggaaggtgcgcaactccgagctgctgagccgcttcaagccgctgctcgatgccggcgacccgcggggccgcgccgcccgcagggaccgcttcaagcagttcgtcaacaacgtggcggtggtgaaggagctcgacggcgtcaagttcgtggtgctgaggaagaagccccggcccccggagcccgagcccgcacccttcggccccccgggggcagcggcccagccgtcgaaacccacttcgacggtcttgccgcggagcgcctctgccccgggagctccgcccttggtccgggtgccgcggccagtggagccgccaggggacctgggtctgccaacagagccacaggacaccccgggggggccggcctccgagcccgctcagccgcccggggagcggtccgccgacccaccgcttccagcccttgagctagcccaggccaccgagagaccctccgcagacgcggccccaccgcctagggccccttctgaggcggcatcgccctgctctgatccgccagacgcggagcccgggcccggggcagcgaaagggccgccgcagcagaagccctgtatgctgccggtgcgctgcgtcccggcccccgccacgctccgcctccgggcggaggagcccggcctgcgccggcagctgtcggaggagccgagcccgcggagctcccctctgctgctgaggcggctctcggtggaagagtccggtctgggcctcggcctgggcccgggccgctccccgcacctgaggcgcctgtcgcgcgccggcccgcgtctgctgagccctgacgccgaggagttgcccgccgcgccgccgccgtccgcggtgcccctggagccgtccgagcacgagtggctcgtgcggactgccgggggccgctggacccaccagctgcacgggctgctgctgcgcgaccgcggcttggcggccaagcgcgacttcatgtctggcttcacggccctgcattgggccgccaagagcggcgacggcgagatggcgctgcagctggtggaggtcgcgcggcgcagtggcgcaccagtcgacgtgaacgcacgctcccacggcggctacacgccgctgcacctggctgcactgcacggccacgaggacgctgccgtgctgctggtggtgcgtctgggtgcccaggtgcacgtgcgtgatcacagcgggcgtcgcgcctaccagtacctgcggcccggctcctcgtacgcgctgcgccgccttcttggcgatccaggcctgcgaggcaccacggagccagatgcgaccggtggtggaagtggcagtcttgctgccaggcgccccgtacaggtggccgccaccatcctcagttccaccaccagtgcatttctgggcgtcctggctgacgacctcatgctccaggacctggcccgaggcttgaagaagtcgagctccttcagcaagttcttgagcgcctcgcccatggctccacgtaaaaagacaaagatccgcggtggtctgccagccttctcagaaatctctcgtcgacctactccggggcctttagctggtctagtgcccagttttcctccaacaacctgaaggtccctggggctaccacctggttgatctatcgtggcctgctcgtcgcagtccaaacccgcccaagactgcagcccaatcaacgcctggagcctcattctgtttccagctttgtccagggcagcctgttttacccagatgggcctgcacctccagcttctttctgaagcatgacccatctccagagatgggatttgagaaatcagtgagagcttcctataaagaactccaggagacaggcctgaactctagagggaacttgaaatccagggtcaaaggttaaggggcagcagctggtctggcccctgaaagccttcacctgatgcctctgcagctacacaatcccaagacagagctagagcaagggtctctttaaatgttgaaagatgttaggtt

SEQ ID NO: 44 - DNA, 398 bp, Homo sapiens
agctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcgtcatgcttatggggatcaagtaagtcatgttggcaataatgtgattttgcatgttttttttttcatggcccagaaatttccaacttgtatgtgttttattcttatcttttggtatctacacccattaagcaaggtatgaaattgagaaatgcatatatgtataactgtatatttacacacatttagct

SEQ ID NO: 45 - DNA, 398 bp, Homo sapiens
agctataaagaagcataatgttggcgtcaaatgtgccactatcactcctgatgagaagagggttgaggagttcaagttgaaacaaatgtggaaatcaccaaatggcaccatacgaaatattctgggtggcacggtcttcagagaagccattatctgcaaaaatatcccccggcttgtgagtggatgggtaaaacctatcatcataggtcatcatgcttatggggatcaagtaagtcatgttggcaataatgtgattttgcatgttttttttttcatggcccagaaatttccaacttgtatgtgttttattcttatcttttggtatctacacccattaagcaaggtatgaaattgagaaatgcatatatgtataactgtatatttacacacatttagct

SEQ ID NO: 46 - DNA, 1192 bp, Homo sapiens
agctccgccctggggaggggtccgcgctgctgattggctgtggccggcaggtgaaccctcagccaatcagcggtacggggggcggtgcctccggggctcacctggctgcagccacgcaccccctctcagtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtaccccggatcccctgacttgcgagggacgcattcgggccgcaagctccgcgccccagccctgcgccccttcctctcccgtcgtcaccgcttcccttcttccaagaaagttcgggtcctgaggagcggagcggcctggaagcctcgcgcgctccggaccccccagtgatgggagtggggggtgggtggtgaggggcgagcgcggctttcctgccccctccagcgcagaccgaggcgggggcgtctggccgcggagtccgcggggtgggctcgcgcgggcggtgggggcgtgaagcggggtgtagggggtggggtgtggagaaggggtgccctggtgcaagtcgagggggagccaggagtcgtggggacgatcttcgagggaaggagaggggcatccgtagaaataaaggcacctgccatgccaagaaaggtcgtaaataggagtgagggtcccggggataagaaagtgaggtcggaggaggtgggagcgcccctcgctctgaggagtggtgcattcccggtctaaggaaagtggggtactggagaataaagacatctccaataaaatgagaaaggagactgaaagggaacggtgggctaggtcttgagggggtgactcggcggccccctcccgggagttcctgggggctcggcggccgtaggtttcggggtgggggagggtgacgtcgctgcccgcccgtcccggggctgcgggctggggtcctcccccaatcccgacgccgggagcgagggaggggcggcgctgttggtttcggtgagcaggagggaaccctccgagtcacccggttccatctacctttcccccaccccaggtctcctcttggctctgccaggagccggagccctgccaccctggctttgacgccgagagc

SEQ ID NO: 47 - DNA, 1101 bp, Homo sapiens
ttaaaaaaatctgttttgttctatgtgattttcccataccaagcaccgtgcccggcacaagctgggatcccagtacacatctcgggacggaagaaccgtgtttccctagaacccagtcagagggcagcttagcaatgtgtcacaggtggggcgcccgcgttccgggcggacgcactggctccccggccggcgtgggtgtggggcgagtgggtgtgtgcggggtgtgcgcggtagagcgcgccagcgagcccggagcgcggagctgggaggagcagcgagcgccgcgcagaacccgcagcgccggcctggcagggcagctcggaggtgggtgggccgcgccgccagcccgcttgcagggtccccattggccgcctgccggccgccctccgcccaaaaggcggcaaggagccgagaggctgcttcggagtgtgaggaggacagccggaccgagccaacgccggggactttgttccctccgcggaggggactcggcaactcgcagcggcagggtctggggccggcgcctgggagggatctgcgccccccactcactccctagctgtgttcccgccgccgccccggctagtctccggcgctggcgcctatggtcggcctccgacagcgctccggagggaccgggggagctcccaggcgcccgggactggagactgatgcatgagggggctacggaggcgcaggagcggtggtgatggtctgggaagcggagctgaagtgccctgggctttggtgaggcgtgacagtttatcatgaccgtgttcaggcaggaaaacgtggatgattactacgacaccggcgaggaacttggcagtggacagtttgcggttgtgaagaaatgccgtgagaaaagcaccggcctccagtatgccgccaaattcatcaagaaaaggaggactaagtccagccggcggggtgtgagccgcgaggacatcgagcgggaggtcagcatcctgaaggagatccagcaccccaatgtcatcaccctgcacgaggtctatgagaacaagacggacgtcatcctgatcttggaactcgttgcaggtggcgagctgtttgacttcttagctgaaaaggaatctttaa

SEQ ID NO: 48 - DNA, 1040 bp, Homo sapiens
ttaaagcaggagaaaggagcagaggaagagaggagatgagagagggagaataaagagagagggaagaagagagagtttgagagatggagaaagagaagacagaaaagagagaagaaaggaaagattttggttgggaaggggtcttccttttctttcccttttcctccttcacttttcctaaaagcagttcctggatctcaaaactgatctctctcggtctgttttccttcctgtctgtccctttccctctttttcctcttctccccctttctccctccctctctcttctgtccgcctccccttctcccctcagcctctcgcccccctcccagtgtccagcccagagtctgcgccccgggcccattgttagcaggctattccacggcagctttgcatctggctccggcgggaagcggaaaacgggaggcggctctcgaagcttcccgaccttcctgcgccatcaacttctcaggagtggctggagaaagatgcatgtgccaagcgagacaacaaccccaggtccatgtgtccaaatccccgtagccagggcggcggccagccaaagaaatgccgccccgagcaggcgcgtgcggctcctggcattctgggtttcatacccgtagggctcgggtgcggtgagtatttccgatttccaggaagtctgttggaagttaccagcagggaaagagaagatgcttgctcctctctttccctccctctctcttttttttctacccttcttcttgtccttgtcgctctggttgggggctggttggtttagatacagcgagtgctccctggcagcctgaactgcctcccacaccttcctgatccttgggcaccggggatgtttgcttccgggttctgttgtgggaaacagcactgcgggggagaggaggcgacccaaggaggaattataaaggttttcgttctccttcattatcttttttccaattcggagacttgagactgagcgcatcttggacagtgctagagggttcaaaagataacacctttaggtaccaaaaaataatttttaa

SEQ ID NO: 49 - DNA, 826 bp, Homo sapiens
ttgggcacaggtgccttagctttgggggcaagggcccgacaaaggtgccaagggctccacttaagacacagatgcctgcagcctggggccccagggaggagcaggccctgtactctgaagctggggccgacgttctggtgctgaggccacgggcagcggcgcagctgccggacagggaggcctggggaccctgtgggtgtggggtcctccctggctgcagctccttggagggctccctctgtccaggctccggctggcgacacaaggggtgtgaggggttcctccctgctgtgactgaggtggtctgagcgggtgtgtgaggcacatgcccggtgatgcccacggcgtgcttcagggcctggttccctggcctgctctccctcaaggacagcccaagggtggtgaatgtctctgtgccatcacctggactgtgcctgctgctgccagctgccgtgttccattctgagtgatcgtggtgcagggacggcgcccacacgcgttgggcccaggctggcctgcctgtgtgctgcggtgctggcttggctcagaacagctcgaggcctcgccgtgaaggctgagggtttctgggcacctgcgtgcgggcgtggccctgctcccccgggtccgtgtctgcgcatcggtctgcactgggcagcgtctgactccgtttctgttctgggcctctgggcctcggcatctgcctgcctcctccgagttcacgcggggcgtggagtgacgggggctgtggcccagccctgcccctgctctgaggctgtgagccatgatccccactcctgtctctttggcagggaatgtctttcacatg

SEQ ID NO: 50 - DNA, 1117 bp, Homo sapiens
ttaaaaagctggttgcgtagattcctgtcaatgctcaggatcctctgccttgtgatatctggagataagtcaacgccttgcaggacgcttacatgctcgggcagtacctctctcagcaacacctccatgcactggtatacaaagtccccctcaccccagccgcgacccttcaaggccaagaggcggcagagcccgaggcctgcacgagcagctctctcttcaggagtgaaggaggccacgggcaagtcgccctgacgcagacgctccaccagggccgcgcgctcgccgtccgccacataccgctcgtagtattcgtgctcagcctcgtagtggcgcctgacgtcgcgttcgcgggtagctacgatgaggcggcgacagaccaggcacagggccccatcgccctccggaggctccaccaccaaataacgctgggtccactcgggccggaaaactagagcctcgtcgacttccatcttgcttcttttgggcgtcatccacattctgcgggaggccacaagagcagggccaacgttagaaaggccgcaaggggagaggaggagcctgagaagcgccaagcacctcctccgctctgcgccagatcacctcagcagaggcacacaagcccggttccggcatctctgctcctattggctggatatttcgtattccccgagctcctaaaaacgaaccaataggaagagcggacagcgatctctaacgcgcaagcgcatatccttctaggtagcgggcagtagccgcttcagggagggacgaagagacccagcaacccacagagttgagaaatttgactggcattcaagctgtccaatcaatagctgccgctgaagggtggggctggatggcgtaagctacagctgaaggaagaacgtgagcacgaggcactgaggtgattggctgaaggcacttccgttgagcatctagacgtttccttggctcttctggcgccaaaatgtcgttcgtggcaggggttattcggcggctggacgagacagtggtgaaccgcatcgcggcgggggaagttatccagcggccagctaatgctatcaaagagatgattgagaactggtacggagggagtcgagccgggctcacttaa

SEQ ID NO: 51 - DNA, 736 bp, Homo sapiens
agctcttgaaattctctttgcaactaggtcgactgtgaaactgcagccagtgtccccaaacgccccaaggataaggcctttatcactccgacgcatcctctctctgctttttaaatagacttttgactcggccaggccccctgccacccaccgcgctgaccctgcctgcgcttgcgtcccgcgtcccgcatccggggggaggcggcgggcccgggtctctggggctggcccatttacttagttttgtttttgttgcatttgcttttggtgacactccctttttgatattccacatataaatgaggcaataattttctttctatgtctgcttatttcacttagcataatggtctccctgtccacctcggctcccgcgcggggaggaggggcaagggccctccgcccgggccccgcctccccgcgcgccgctgccaccgccccgcacctttttttgactttttaataatgctcattctgactgtgataaggtggtatctcattatggttttaattggtatttcccttgtgattggtgatgttgagtatttttcatgtttcttggccatttgtgtatcttcttttgaaaaatgtccgtttggctgggcgcggtggctcacgtctgtaatcctagcactttcggaagccaaggcgggtggatcacctgaggtcaggagttcgagaccagcctggctaacatggtgaaactccgtgtctactaaaaatacaaaaattagct

SEQ ID NO: 52 - DNA, 898 bp, Homo sapiens
agcttccaggtgtgtctcgtgaactgcacaagcatttttttgtccccatgcgtcctacctggcagtacaggctggtcacgcacgccctgtaagaccagtggccagcctccatactttctgtcacaccttcaaagtgacaccaacttatgttatcagcctattaatttagcaaagttaaaagggataaaaaataacaaagccttcagagtgacagattataccgaatttattgcataaaagggtcagtaagcacccgcgggtctcgaaccccagcacagagatggacagatagaaagtccggcatgaggaggtaaccgcttgagctaactccgacccgggtaggagtgtgcgactgaaacttctaaaccatagaagagtgacgatgtggagagggacgggctgcatgtgctccccgcccccgagaggcctgcgtcatgcggtctcgcccgctctgcgccaggcgtcctgctaacgtgtcctggagggactctcagttcctcccgcccgcatcctgcgcgggaaccgtggaagggggcaaaatccacccactggaggggaggcaggagggtgcgggggggcgtgtgggccgtctacctaggtccagcagccaggctgctgaggagtacccccgccaaaggcttttcggggttctctccagcagacgggggcagcctaaggcctccataaaatctccccgaagcagcctatgaacttgccaaaggcaggggttctgtggtttcatttcttgccaggtcctgggctggaccctggagacggagttcatcatcagcaaatcctctttgggtggggagttgaatgaattaatgagtgattagcagggcactagggcccaggagtaagcccagaattgagatcacaggctgaagatttaaaagct